(19) United States
(12) Patent Application Publication
(10) Pub. No.: US 2018/0320128 A1
(43) Pub. Date: Nov. 8, 2018

(54) METHODS FOR MODULATING PRODUCTION PROFILES OF RECOMBINANT PROTEINS

(71) Applicant: ARES TRADING S.A., AUBONNE (CH)

(72) Inventors: DAVID BRUHLMANN, LAUSANNE (CH); MARTIN JORDAN, ECUBLENS (CH)

(21) Appl. No.: 15/773,186

(22) PCT Filed: Nov. 9, 2016
(86) PCT No.: PCT/EP2016/077159

§ 371 Date: May 3, 2018

(30) Foreign Application Priority Data
Nov. 11, 2015 (EP) 15194105.1

(51) Int. Cl. C12N 1/38; C12N 15/85; C12N 5/071; C12N 5/00; C07K 19/00

(52) U.S. Cl. CPC C12N 1/38; C12N 15/85; C12N 5/0682; C12P 21/005; C07K 19/00; C12N 2015/8518; C12N 2500/34; C12N 5/0037

(57) ABSTRACT

The present invention relates to methods and compositions for modulating glycosylation of recombinant proteins expressed by mammalian host cells during the cell culture process. Also disclosed are methods of culturing a host cell expressing a recombinant protein in a cell culture medium comprising a disaccharide or a trisaccharide, while keeping the osmolality constant.

### FIELD OF THE INVENTION

The present invention relates to methods and compositions for modulating glycosylation of recombinant proteins expressed by mammalian host cells during the cell culture process. Also disclosed are methods of culturing a host cell expressing a recombinant protein in a cell culture medium comprising a disaccharide or a trisaccharide, while keeping the osmolality constant.

### BACKGROUND OF THE INVENTION

The glycosylation profile of a protein, such as a therapeutic protein or an antibody, is an important characteristic that influences biological activity of the protein through changes in half-life and affinity due to effects for instance on folding, stability and antibody-dependent cellular cytotoxicity (ADCC, one of the mechanism responsible for the therapeutic effect of antibodies). Glycosylation is highly dependent on the cell line that is used for the production of the protein of interest, as well as on the cell culture processes (pH, temperature, cell culture media composition, raw material lot-to-lot variation, medium filtration material, air, etc).

ADCC activity is influenced by the amount of fucose and/or mannose linked to the oligosaccharides of the Fc region, with enhanced activity seen with a reduction in fucose and/or an increase in mannose. Indeed, for instance, compared to fucosylated IgGs, non-fucosylated forms exhibit dramatically enhanced ADCC due to the enhancement of FcγRIIIa binding capacity without any detectable change in complement-dependent cytotoxicity (CDC) or antigen binding capability (Yamane-Ohnuki and Satoh, 2009). Similarly, antibodies exhibiting high level of mannose-5 glycans also presented higher ADCC (Yu et al., 2012). Thus, where the ADCC response is the principle therapeutic mechanism of antibody activity, the provision of methods for the preparation of recombinant therapeutic protein with a glycosylation profile characterized by decreased fucosylation and/or increased mannosylation, are beneficial. The advantages of non-fucosylated and/or highly mannosylated antibodies also include achieving therapeutic efficacy at low doses. However, many therapeutic antibodies that are currently on the market are heavily fucosylated because they are produced by mammalian cell lines with intrinsic enzyme activity responsible for the core-fucosylation of the Fc N-glycans of the products.

Modulation of protein glycosylation is of particular relevance for marketed therapeutic proteins or antibodies as glycosylation (such as mannosylation and/or fucosylation) can impact therapeutic utility and safety. Further, in the frame of biosimilar compounds, control of the glycosylation profile of a recombinant protein is crucial, as the glycosylation profile of said recombinant protein has to be comparable to the glycosylation profile of the reference product.

Optimisation of culture conditions to obtain the greatest possible productivity is one of the other main aims of recombinant protein production. Even marginal increases in productivity can be significant from an economical point of view. Many commercially relevant proteins are produced recombinantly in host cells. This leads to a need to produce these proteins in an efficient and cost effective manner. Unfortunately, one of the drawback of recombinant protein production is that the conditions in which cell culture is performed usually favors a reduction of cell viability over time, reducing both efficiency and overall productivity.

Perfusion culture, Batch culture and Fed batch culture are the basic methods for culturing animal cells for producing recombinant proteins. Very often, especially in fed-batch and perfusion methods, inducing agents are added to the culture media to increase production of proteins in cells. These inducers induce the cell to produce more desired product. One such agent is sodium butyrate. However, the drawback of using sodium butyrate in cell culture is that it affects significantly cell viability. For instance Kim et al (2004) have shown that although sodium butyrate was able to increase protein production in recombinant CHO cells in a batch culture, at the end of the production run (after 8 days of culture), cell viability was less than 45%. Repeating the same experiments in perfusion batch culture, the authors noticed that within 6 days of treatment, cell viability was as low as 15%.

Although the use of an inducer can increase protein production, the drawback concerning cell viability has to be considered. Indeed, the use of a well-known inducer, such as sodium butyrate, can be counterproductive after about 5 days in culture, whereas a typical production period is between 12 to 15 days in fed-batch mode and can be up to 40-45 days in perfusion mode.

Because many proteins are recombinantly produced by cells grown in culture for more than 6 days, there is a need for methods allowing more efficient production runs, while maintaining acceptable cell viability over a longer time.

There also remains a need for culture conditions and production methods allowing not only for increased recombinant protein productivity by maintaining high cell density, increasing the harvest titre or avoiding substantial decrease in cell viability over a production period but also for controling the glycosylation profile, such as fucosylation and/or mannosylation profiles, of a recombinant protein. The present invention addresses these needs by providing methods and compositions for increasing production of recombinant proteins and/or for modulating recombinant protein glycosylation without negative impact on efficiency on the production.

### SUMMARY OF THE INVENTION

In one aspect the invention provides a method of producing a recombinant protein in fed-batch or batch mode, said method comprising culturing a mammalian host cell expressing said recombinant protein in a cell culture medium comprising a dissacharide or a trisaccharide, or supplemented with a dissacharide or a trisaccharide, while maintaining the osmolality similar to the one of a standard medium which does not comprise said disaccharide or trisaccharide.

In another aspect, here is disclosed a method of culturing in fed-batch or batch mode a mammalian host cell that expresses a recombinant protein, said method comprising culturing said host cell in a cell culture medium comprising a dissacharide or a trisaccharide, or supplemented with a dissacharide or a trisaccharide, while maintaining the osmolality similar to the one of a standard medium which does not comprise said disaccharide or trisaccharide.

In a further aspect, the invention provides a method of increasing production of a recombinant protein in fed-batch or batch mode, said method comprising culturing a mammalian host cell expressing said protein in a cell culture medium comprising a dissacharide or a trisaccharide, or supplemented with a dissacharide or a trisaccharide, while maintaining the osmolality similar to the one of a standard medium which does not comprise said disaccharide or trisaccharide.

In another aspect, here is disclosed a method of producing a recombinant protein with a modulated glycosylation profile, said method comprising culturing a host cell expressing said protein in cell culture medium comprising a disaccharide or a trisaccharide or supplemented with a disaccharide or a trisaccharide, while maintaining the osmolality of the culture medium similar to the one of a standard medium which does not comprise said disaccharide or trisaccharide.

In a even further aspect, the invention provides a method of producing a recombinant protein with a modulated glycosylation profile, said method comprising culturing a host cell expressing said protein in cell culture medium complemented with at least one feed comprising a disaccharide or a trisaccharide while maintaining the osmolality of the culture medium similar to the one of a standard medium which does not comprise said disaccharide or trisaccharide

In still a further aspect, the invention provides use of a trisaccharide as an inducer and/or to improve the efficiency or production run.

According to the invention, the disaccharide is preferably sucrose and the trisaccharide is preferably raffinose.

### BRIEF DESCRIPTION OF THE FIGURES

FIG. 1 is a schema of experimental approach 1 with constant osmolality and increasing sugar concentration (see example 1). Black bars=concentration of sodium chloride, grey bars=concentration of sugar.

FIG. 2 shows the effect on mAb1 cells of various concentrations of raffinose, at constant osmolality (315 mOsm/kg). a. Growth profile and b. viability shown from mAb1 cells expressing mAb1, cultivated in 96 deep-well plates for 14 days. Samples for viable cell density and viability (Guava) were taken at working days 3, 5, 7, 10, 12 and 14.

FIG. 3 shows the effect on mAb1/mAb1 cells of various concentrations of raffinose, at constant osmolality (315 mOsm/kg). a. absolute harvest titer on working day 14, b. specific productivity on working day 14 [pg/cell/day], c. absolute change in glycosylation with respect to control shown from mAb1 cells expressing mAb1; Unknown=unknown, Gal=galactosylated, Man=High Mannose, Sial=sialylated, Non Fuc=non fucosylated, Fuc=fucosylated glycoforms.

FIG. 4 shows the effect on mAb2 cells of various concentrations of raffinose, at constant osmolality (315 mOsm/kg). a. Growth profile and b. viability shown from mAb2 cells expressing mAb2, cultivated in 96 deep-well plates for 14 days. Samples for Viable Cell Density and viability (Guava) were taken at working days 3, 5, 7, 10, 12 and 14.

FIG. 5 shows the effect on mAb2/mAb2 cells of various concentrations of raffinose, at constant osmolality (315 mOsm/kg). a. absolute harvest titer on working day 14, b. specific productivity [pg/cell/day], c. absolute change in glycosylation with respect to control shown from mAb2 cells expressing mAb2; Unknown=unknown, Gal=galactosylated, Man=High Mannose, Sial=sialylated, Non Fuc=non-fucosylated, Fuc=fucosylated glycoforms

FIG. 6 shows the effect on mAb1 cells of various concentrations of sucrose, at constant osmolality (315 mOsm/kg). a. Growth profile and b. viability shown from mAb1 cells expressing mAb1, cultivated in 96 deep-well plates for 14 days. Samples for viable cell density and viability (Guava) were taken at working days 3, 5, 7, 10, 12 and 14.

FIG. 7 shows the effect on mAb1/mAb1 cells of various concentrations of sucrose, at constant osmolality (315 mOsm/kg). a. relative harvest titer on working day 14, b. specific productivity [pg/cell/day], c. absolute change in glycosylation with respect to control, shown from mAb1 cells expressing mAb1; Unknown=unknown, Gal=galactosylated, Man=High Mannose, Sial=sialylated, Non Fuc=non fucosylated, Fuc=fucosylated glycoforms.

FIG. 8 shows the effect on mAb2 cells of various concentrations of sucrose, at constant osmolality (315 mOsm/kg). Growth profile (a) and viability (b) shown from mAb2 cells expressing mAb2, cultivated in 96 deep-well plates for 14 days. Samples for Viable Cell Density and viability (Guava) were taken at working days 3, 5, 7, 10, 12 and 14.

FIG. 9 shows the effect on mAb2/mAb2 cells of various concentrations of sucrose, at constant osmolality (315 mOsm/kg). a. absolute harvest titer on working day 14, b. specific productivity [pg/cell/day], c. absolute change in glycosylation with respect to control shown from mAb2 cells expressing mAb2; Unknown=unknown, Gal=galactosylated, Man=High Mannose, Sial=sialylated, Non Fuc=non-fucosylated, Fuc=fucosylated glycoforms.

FIG. 10 shows the effect on mAb1 cells of various concentrations of raffinose, at constant osmolality (315 mOsm/kg). a. Growth profile, b. viability of mAb1 cells expressing mAb1, cultivated in Spin Tubes for 14 days, Samples for Viable Cell Density and viability (ViCell) were taken at working days 3, 5, 7, 10, 12 and 14, n=2

FIG. 11 shows the effect on mAb1 cells of various concentrations of raffinose, at constant osmolality (315 mOsm/kg). a. absolute harvest titer on WD 14 (Biacore) b. specific cell productivity per day [pg/cell/day] of mAb1 cells expressing mAb1, cultivated in Spin Tubes for 14 days. Samples were taken at working days 5, 7, 10, 12 and 14, n=2

FIG. 12 shows the effect on mAb1 glycosylation of various concentrations of raffinose, at constant osmolality (absolute change in glycosylation with respect to control shown from mAb1 cells expressing mAb1) (315 mOsm/kg). Unknown=unknown, Gal=galactosylated, Man=High Mannose, Sial=sialylated, Non Fuc=non-fucosylated, Fuc=fucosylated glycoforms

FIG. 13 shows the effect on mAb2 cells of two concentration of raffinose (0 or 30 mM), at various osmolalities. a. Growth profile and b. viability shown from mAb2 cells expressing mAb2, cultivated in 96 deep-well plates for 14 days. Samples for viable cell density and viability (Guava) were taken at working days 3, 5, 7, 10, 12 and 14. Supplementation of raffinose in medium is labeled with “30 mM raffinose” (empty symbols)

FIG. 14 shows the effect on mAb2/mAb2 cells of two concentration of raffinose (0 or 30 mM), at various osmolalities. a. absolute harvest titer on WD14, b. specific productivity [pg/cell/day]. and c. absolute change in glycosylation with respect to control shown from mAb2 cells expressing mAb2; Unknown=unknown, Gal=galactosylated, Man=High Mannose, Sial=sialylated, Non Fuc=non fucosylated, Fuc=fucosylated glycoforms. Supplementation of raffinose in medium is labeled with “30 mM raffinose” (shown dashed)

### DETAILED DESCRIPTION OF THE INVENTION

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

Unless defined otherwise, all technical and scientific terms used herein have the same meaning as is commonly understood by one of skill in art to which the subject matter herein belongs. As used herein, the following definitions are supplied in order to facilitate the understanding of the present invention.

As used in the specification and claims, the term “and/or” used in a phrase such as “A and/or B” herein is intended to include “A and B”, “A or B”, “A”, and “B”.

The abbreviation “WD” that can be used in the description as a whole and in the figures stands for working day.

As used in the specification and claims, the term “cell culture” or “culture” is meant the growth and propagation of cells in vitro, i.e. outside of an organism or tissue. Suitable culture conditions for mammalian cells are known in the art, such as taught in Cell Culture Technology for Pharmaceutical and Cell-Based Therapies (2005). Mammalian cells may be cultured in suspension or while attached to a solid substrate.

The terms “cell culture medium,” “culture medium”, “medium,” and any plural thereof, refer to any medium in which cells of any type can be cultured. A “basal medium” refers to a cell culture medium that contains all of the essential ingredients useful for cell metabolism. This includes for instance amino acids, lipids, carbon source, vitamins and mineral salts. DMEM (Dulbeccos' Modified Eagles Medium), RPMI (Roswell Park Memorial Institute Medium) or medium F12 (Ham's F12 medium) are examples of commercially available basal media. Alternatively, said basal medium can be a proprietary medium fully developed in-house, also herein called “chemically defined medium” or “chemically defined culture medium”, in which all of the components can be described in terms of the chemical formulas and are present in known concentrations. The culture medium can be free of proteins and/or free of serum, and can be supplemented by any additional standard compound(s) such as amino acids, salts, sugars, vitamins, hormones, growth factors, depending on the needs of the cells in culture.

The term “standard medium” refers to a cell culture medium having an osmolality comprised between 300 and 330 mOsm/kg, preferably at or at about 315 mOsm/kg. According to the present invention, the term “standard medium” is used for a medium that does not comprise a disaccharide or a trisaccharide, but which is otherwise completely similar in terms of components to the culture medium comprising the disaccharide or the trisaccharide. For instance if one uses the standard medium “A”, the only differences with a medium “A′” will be the presence of a disaccharide such as sucrose or of a trisaccharide such as raffinose and possibly the concentration in salt (as the osmolality according to the invention is kept constant by varying the concentration in salt).

The term “feed medium” (and plural thereof) refers to a medium used as a supplementation during culture to replenish the nutrients which are consumed. The feed medium can be a commercially available feed medium or a proprietary feed medium (herein alternatively chemically defined feed medium).

The term “bioreactor” or “culture system” refers to any system in which cells can be cultured, preferably in batch or fed-batch mode. This term includes but is not limited to flasks, static flasks, spinner flasks, tubes, shake tubes, shake bottles, wave bags, bioreactors, fiber bioreactors, fluidized bed bioreactors, and stirred-tank bioreactors with or without microcarriers. Alternatively, the term “culture system” also includes microtiter plates, capillaries or multi-well plates. Any size of bioreactor can be used, for instance from 0.1 milliliter (0.1 mL, very small scale) to 20000 liters (20000 L or 20 KL, large scale), such as 0.1 mL, 0.5 mL 1 mL, 5 mL, 0.01 L, 0.1 L, 1 L, 2 L, 5 L, 10 L, 50 L, 100 L, 500 L, 1000 L (or 1 KL), 2000 L (or 2K), 5000 L (or 5 KL), 10000 L (or 10 KL), 15000 L (or 15 KL) or 20000 L (20 KL).

The term “fed-batch culture” refers to a method of growing cells, where there is a bolus or continuous feed media supplementation to replenish the nutrients which are consumed. This cell culture technique has the potential to obtain high cell densities in the order of greater than 10×10⁶ to 30×10⁶ cells/nil, depending on the media formulation, cell line, and other cell growth conditions. A biphasic culture condition can be created and sustained by a variety of feed strategies and media formulations.

Alternatively a perfusion culture can be used. Perfusion culture is one in which the cell culture receives fresh perfusion feed medium while simultaneously removing spent medium. Perfusion can be continuous, step-wise, intermittent, or a combination of any or all of any of these. Perfusion rates can be less than a working volume to many working volumes per day. Preferably the cells are retained in the culture and the spent medium that is removed is substantially free of cells or has significantly fewer cells than the culture. Perfusion can be accomplished by a number of cell retention techniques including centrifugation, sedimentation, or filtration (see for example Voisard et al., 2003). When using the methods and/or cell culture techniques of the instant invention, the proteins are generally directly secreted into the culture medium. Once said protein is secreted into the medium, supernatants from such expression systems can be first concentrated using a commercially available protein concentration filter.

The efficiency of a production run is measured for instance by an increase of the viable cell density, a lower decrease in cell viability and/or higher harvest titre.

As used herein, “cell density” refers to the number of cells in a given volume of culture medium. “Viable cell density” (VCD) refers to the number of live cells in a given volume of culture medium, as determined by standard viability assays. The terms “Higher cell density” or “Higher viable cell density”, and equivalents thereof, means that the cell density or viable cell density is increased by at least 15% when compared to the control culture condition. The cell density will be considered as maintained if it is in the range of −15% to 15% compared to the control culture condition. The terms “Lower cell density” or “Lower viable cell density”, and equivalents thereof, means that the cell density or viable cell density is decreased by at least 15% when compared to the control culture condition.

The term “viability”, or “cell viability” refers to the ratio between the total number of viable cells and the total number of cells in culture. Viability is usually acceptable as long as it is at not less than 50% compared to the start of the culture. Viability is often used to determine time for harvest. For instance, in fed-batch culture, harvest can be performed once viability reaches at 50% or after 14 days in culture.

The wording “titre” refers to the amount or concentration of a substance, here the protein of interest, in solution. In the context of the invention it is also referred to as harvest titre (titre at the time of after harvest). It is an indication of the number of times the solution can be diluted and still contain detectable amounts of the molecule of interest. It is calculated routinely for instance by diluting serially (1:2, 1:4, 1:8, 1:16, etc) the sample containing the protein of interest and then using appropriate detection method (colorimetric, chromatographic etc.), each dilution is assayed for the presence of detectable levels of the protein of interest. Titre can also be measured by means such as by fortéBIO Octet® or with Biacore C®, as used in the example section.

The term “specific productivity” refers to the amount of a substance, here the protein of interest, produced per cell per day.

The terms “higher titre” or “higher specific productivity”, and equivalents thereof, means that the titre or the productivity is increased by at least 10% when compared to the control culture condition. The titre or specific productivity will be considered as maintained if it is in the range of −10% to 10% compared to the control culture condition. The terms “lower titre” or “lower productivity”, and equivalents thereof, means that the titre or the productivity is decreased by at least 10% when compared to the control culture condition.

The term “osmolality” refers to the total concentration of solved particles in a solution and is specified in osmoles of solute in a kilogram of solvent. It is usually expressed as mOsm/kg.

As used in the specification and claims, a “modulated glycosylation profile” includes a glycosylation profile of a recombinant protein (for example a therapeutic protein or antibody) that is modulated as compared to the glycosylation profile of that same protein produced by culturing a recombinant cell expressing that recombinant protein in a standard culture medium which is not supplemented with a disaccharide, such as sucrose, or trisaccharide, such as raffinose. The modulated glycosylation profile may include modulation of a fucosylation level and/or a mannosylation level in said protein. In an embodiment, the modulated glycosylation profile may include an overall increase in the level of mannosylation and an overall decrease in the level of fucosylation of the protein.

The term “protein” as used herein includes peptides and polypeptides and refers to compound comprising two or more amino acid residues. A protein according to the present invention includes but is not limited to a cytokine, a growth factor, a hormone, a fusion protein, an antibody or a fragment thereof. A therapeutic protein refers to a protein that can be used or that is used in therapy.

The term “recombinant protein” means a protein produced by recombinant technics. Recombinant technics are well within the knowledge of the skilled person (see for instance Sambrook et al., 1989, and updates).

As used in the specification and claims, the term “antibody”, and its plural form “antibodies”, includes, inter alia, polyclonal antibodies, affinity-purified polyclonal antibodies, monoclonal antibodies, and antigen-binding fragments, such as F(ab′)2, Fab proteolytic fragments, and single chain variable region fragments (scFvs). Genetically engineered intact antibodies or fragments, such as chimeric antibodies, scFv and Fab fragments, as well as synthetic antigen-binding peptides and polypeptides, are also included.

The term “humanized” immunoglobulin refers to an immunoglobulin comprising a human framework region and one or more CDRs from a non-human (usually a mouse or rat) immunoglobulin. The non-human immunoglobulin providing the CDRs is called the “donor” and the human immunoglobulin providing the framework is called the “acceptor” (humanization by grafting non-human CDRs onto human framework and constant regions, or by incorporating the entire non-human variable domains onto human constant regions (chimerization)). Constant regions need not be present, but if they are, they must be substantially identical to human immunoglobulin constant regions, i.e., at least about 85-90%, preferably about 95% or more identical. Hence, all parts of a humanized immunoglobulin, except possibly the CDRs and a few residues in the heavy chain constant region if modulation of the effector functions is needed, are substantially identical to corresponding parts of natural human immunoglobulin sequences. Through humanizing antibodies, biological half-life may be increased, and the potential for adverse immune reactions upon administration to humans is reduced.

As used in the specification and claims, the term “fully human” immunoglobulin refers to an immunoglobulin comprising both a human framework region and human CDRs. Constant regions need not be present, but if they are, they must be substantially identical to human immunoglobulin constant regions, i.e., at least about 85-90%, preferably about 95% or more identical. Hence, all parts of a fully human immunoglobulin, except possibly few residues in the heavy chain constant region if modulation of the effector functions or pharmacokinetic properties are needed, are substantially identical to corresponding parts of natural human immunoglobulin sequences. In some instances, amino acid mutations may be introduced within the CDRs, the framework regions or the constant region, in order to improve the binding affinity and/or to reduce the immunogenicity and/or to improve the biochemical/biophysical properties of the antibody.

The term “recombinant antibodies” means antibodies produced by recombinant technics. Because of the relevance of recombinant DNA techniques in the generation of antibodies, one needs not be confined to the sequences of amino acids found in natural antibodies; antibodies can be redesigned to obtain desired characteristics. The possible variations are many and range from the changing of just one or a few amino acids to the complete redesign of, for example, the variable domain or constant region. Changes in the constant region will, in general, be made in order to improve, reduce or alter characteristics, such as complement fixation (e.g. complement dependent cytotoxicity, CDC), interaction with Fc receptors, and other effector functions (e.g. antibody dependent cellular cytotoxicity, ADCC), pharmacokinetic properties (e.g. binding to the neonatal Fc receptor; FcRn). Changes in the variable domain will be made in order to improve the antigen binding characteristics. In addition to antibodies, immunoglobulins may exist in a variety of other forms including, for example, single-chain or Fv, Fab, and (Fab′)2, as well as diabodies, linear antibodies, multivalent or multispecific hybrid antibodies.

As used herein, the term “antibody portion” refers to a fragment of an intact or a full-length chain or antibody, usually the binding or variable region. Said portions, or fragments, should maintain at least one activity of the intact chain/antibody, i.e. they are “functional portions” or “functional fragments”. Should they maintain at least one activity, they preferably maintain the target binding property. Examples of antibody portions (or antibody fragments) include, but are not limited to, “single-chain Fv”, “single-chain antibodies,” “Fv” or “scFv”. These terms refer to antibody fragments that comprise the variable domains from both the heavy and light chains, but lack the constant regions, all within a single polypeptide chain. Generally, a single-chain antibody further comprises a polypeptide linker between the VH and VL domains which enables it to form the desired structure that would allow for antigen binding. In specific embodiments, single-chain antibodies can also be bi-specific and/or humanized.

A “Fab fragment” is comprised of one light chain and the variable and CH1 domains of one heavy chain. The heavy chain of a Fab molecule cannot form a disulfide bond with another heavy chain molecule. A “Fab′ fragment” that contains one light chain and one heavy chain and contains more of the constant region, between the CH1 and CH2 domains, such that an interchain disulfide bond can be formed between two heavy chains is called a F(ab′)2 molecule. A “F(ab′)2” contains two light chains and two heavy chains containing a portion of the constant region between the CH1 and CH2 domains, such that an interchain disulfide bond is formed between two heavy chains. Having defined some important terms, it is now possible to focus the attention on particular embodiments of the instant invention.

Examples of known antibodies which can be produced according to the present invention include, but are not limited to, adalimumab, alemtuzumab, belimumab, bevacizumab, canakinumab, certolizumab pegol, cetuximab, denosumab, eculizumab, golimumab, infliximab, natalizumab, ofatumumab, omalizumab, pertuzumab, ranibizumab, rituximab, siltuximab, tocilizumab, trastuzumab, ustekinumab or vedolizomab.

Most naturally occurring proteins comprise carbohydrate or saccharide moieties attached to the peptide via specific linkages to a select number of amino acids along the length of the primary peptide chain. Thus, many naturally occurring peptides are termed “glycopeptides” or “glycoproteins” or are referred to as “glycosylated” proteins or peptides. The predominant sugars found on glycoproteins are fucose, galactose, glucose, mannose, N-acetylgalactosamine (“GalNAc”), N-acetylglucosamine (“GlcNAc”), and sialic acid. The oligosaccharide structure attached to the peptide chain is known as a “glycan” molecule. The nature of glycans impact the tridimensional structure and the stability of the proteins on which they are attached. The glycan structures found in naturally occurring glycopeptides are divided into two main classes: “N-linked glycans” or N-linked oligosaccharides” (main form in eukaryotic cells) and “O-linked glycans” or O-linked oligosaccharides”. Peptides expressed in eukaryotic cells typically comprise N-glycans. The processing of the sugar groups for N-linked glycoproteins occurs in the lumen of the endoplasmic reticulum (ER) and continues in the Golgi apparatus. These N-linked glycosylations occur on asparagine residue in the peptide primary structure, on sites containing the amino acid sequence asparagine-X-serine/threonine (X is any amino acid residue except proline and aspartic acid).

Main glycans that can be found on the antibody or fragments thereof secreted by CHO cells are presented in Table 1:

“Glycoform” refers to an isoform of a protein, such as an antibody or a fragment thereof, differing only in the number and/or type of attached glycans. Usually, a composition comprising a glycoprotein comprises a number of different glycoforms of said glycoprotein.

Techniques for the determination of glycan primary structure are well known in the art and are described in detail, for example, in Roth et al. (2012) or Song et al. (2014) It is routine to isolate proteins produced by a cell and to determine the structure(s) of their N-glycans. N-glycans differ with respect to the number of branches (also called “antennae”) comprising sugars, as well as in the nature of said branch(es), which can include in addition to the man3GlcNac2 core structure for instance N-acetylglucosamine, galactose, N-acetylgalactosamine, N-acetylneuraminic acid, fucose and/or sialic acid. For a review of standard glycobiology nomenclature see Essentials of Glycobiology, 1999.

Fucosylated proteins comprise at least one residue of fucose and include for instance glycans such as G0F, G1F and/or G2F (see Table 1).

The N-glycans structures on proteins comprise at least three residues of mannose. These structures can be further mannosylated. The mannosylated glycans such as Man5, Man6 or Man7 are called high-mannose glycans (see Table 1).

The term “subject” is intended to include (but not limited to) mammals such as humans, dogs, cows, horses, sheep, goats, cats, mice, rabbits, or rats. More preferably, the subject is a human.

The terms “Inducing agent”, “inducer” or “productivity enhancer” refer to a compound or a composition (such a culture medium) allowing an increase of the production performance or of the protein production when added in cell cultures. For instance, one of the inducers known for E. coli production is IPTG (Isopropyl β-D-1-thiogalactopyranoside) and inducers for CHO production are among others sodium butyrate, doxycycline or dexamethasone.

The present invention provides methods and compositions for increasing the efficiency of production runs and/or modulating the glycosylation profile of a recombinant protein such as therapeutic protein or antibody. The present invention is based on the optimization of cell culture conditions for protein manufacturing, such as production of antibodies or antigen-binding fragments, resulting in more efficient production runs and/or in the production of a recombinant protein with modulated glycosylation profiles, preferably with decreased fucosylation and/or increased mannosylation (i.e. an increase in high-mannose glycans, such as Man5), without negatively impacting efficiency of the production.

It was surprisingly shown that under cell culture conditions supplemented with a disaccharide such as sucrose or a trisaccharide such as raffinose (which are not standard components of a culture medium or a feed medium), and controlling as well the osmolality of the culture medium, the high mannosylated glycoform content of the recombinant protein and/or the fucosylated glycoform of the recombinant protein can be modulated. Thus during the cell culture production run, when it is desirable to modulate glycosylation profile of a recombinant protein, such as a fucosylation level and/or a mannosylation level in the recombinant protein being produced, the cell culture can be fed with a cell culture medium supplemented with a disaccharide such as sucrose or a trisaccharide such as raffinose, while acting on the osmolality, preferably keeping it constant compared to a standard medium which does not comprise said disaccharide or said trisaccharide (i.e. keeping it or maintaining it similar to the one of a standard medium which does not comprise said disaccharide or said trisaccharide). Alternatively, the cell culture medium can already comprise said disaccharide or trisaccharide, as long as the osmolality of said culture medium is maintained similar to the one of a standard medium which does not comprise said disaccharide or said trisaccharide. It was also shown that under cell culture conditions supplemented with a disaccharide or a trisaccharide, while keeping the osmolality constant compared to a standard medium which does not comprise said disaccharide or said trisaccharide, more efficient run could be achieved (eg. higher VCD and/or cell viability and/or overall titre).

### D-(+)-Raffinose (Herein Raffinose): (O-α-D-Galactopyranosyl-(1→6)-α-D-g lucopyranosyl-(1→2)-β-D-fructofuranoside)

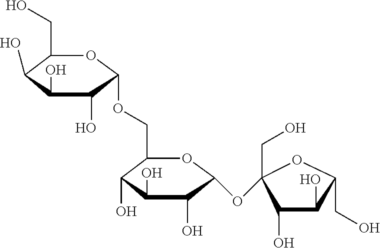

### D-(+)-Sucrose (Herein Sucrose): α-D-glucopyranosyl-(1→2)-β-D-fructofuranoside

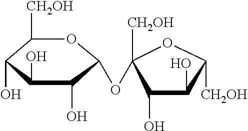

In one aspect the invention provides a method of producing a recombinant protein in fed-batch or batch mode, said method comprising culturing a mammalian host cell expressing said recombinant protein in a cell culture medium comprising a dissacharide or a trisaccharide, or supplemented with a dissacharide or a trisaccharide, while maintaining the osmolality similar to the one of a standard medium which does not comprise said disaccharide or trisaccharide. In some preferred embodiments, the disaccharide is sucrose and the trisaccharide is raffinose.

Alternatively, the present invention describes a method of culturing in fed-batch or batch mode a mammalian host cell that expresses a recombinant protein, said method comprising culturing said host cell in a cell culture medium comprising a dissacharide or a trisaccharide, or supplemented with a dissacharide or a trisaccharide, while maintaining the osmolality similar to the one of a standard medium which does not comprise said disaccharide or trisaccharide. In some preferred embodiments, the disaccharide is sucrose and the trisaccharide is raffinose.

In a further aspect the invention provides a method of increasing production of a recombinant protein in fed-batch or batch mode, said method comprising culturing a mammalian host cell expressing said protein in a cell culture medium comprising a dissacharide or a trisaccharide, or supplemented with a dissacharide or a trisaccharide, while maintaining the osmolality similar to the one of a standard medium which does not comprise said disaccharide or trisaccharide. In some preferred embodiments, the disaccharide is sucrose and the trisaccharide is raffinose.

In an even further aspect the invention provides the use of a disaccharide or a trisaccharide in a cell culture medium, while maintaining the osmolality of the resulting culture medium similar to the one of a standard medium, as an inducer and/or to improve the efficiency of at least one production run.

In another aspect, the invention provides a method of producing a recombinant protein with a modulated glycosylation profile, said method comprising culturing a host cell expressing said protein in cell culture medium comprising a disaccharide or a trisaccharide or supplemented with a disaccharide or a trisaccharide, while maintaining the osmolality of the culture medium similar to the one of a standard medium which does not comprise said disaccharide or trisaccharide. In some preferred embodiments, the disaccharide is sucrose and the trisaccharide is raffinose.

In still a further aspect, herein described is a method of producing a recombinant protein with a modulated glycosylation profile, said method comprising culturing a host cell expressing said protein in cell culture medium complemented with at least one feed comprising a disaccharide or a trisaccharide while maintaining the osmolality of the culture medium similar to the one of a standard medium which does not comprise said disaccharide or trisaccharide. In some preferred embodiments, the disaccharide is sucrose and the trisaccharide is raffinose.

Preferably, in the context of the invention as a whole, the modulated glycosylation profile of the protein comprises modulation of the fucosylation level and/or of the mannosylation level in said protein. In particular, the modulation of the fucosylation level is a decrease in the overall fucosylation level in the recombinant protein and/or the modulation of the mannosylation level is an increase in the overall mannosylation level in the recombinant protein. More particularly the decrease in fucosylation level is due at least to a decrease in G0F and/or GIF forms, even more particularly the decrease in fucosylation level is due at least to a decrease in G0F form. More particularly the increase in mannosylation level is due at least to an increase in high-mannose forms, such as Man5. Preferably, the overall fucosylation level is decreased by about 0.1% to about 99% such as about 0.1%, 1%, 1.5%, 2%, 2.5%, 3%, 3.5%, 4%, 4.5%, 5%, 6%, 7%, 8%, 9%, 10%, 15%, 20%, 25%, 30%, 35%, 40%, 45%, 50%, 51%, 52%, 53%, 54%, 55%, 60%, 65%, 70%, 75%, 80%, 85%, 90%, 95%, or 99%. Should the fucosyl residues completely disappear, the protein will be afucosylated. In another embodiment, the overall mannosylation amount or level is increased by about 0.1% to about 100% such as about 0.1%, 1%, 1.5%, 2%, 2.5%, 3%, 3.5%, 4%, 4.5%, 5%, 6%, 7%, 8%, 9%, 10%, 15%, 20%, 25%, 30%, 35%, 40%, 45%, 50%, 51%, 52%, 53%, 54%, 55%, 60%, 65%, 70%, 75%, 80%, 85%, 90%, 95%, 99% or 100%. Alternatively both modifications occur at the same time, i.e decrease of fucosylation and increase of mannosylation.

As used herein, the phrase “cell viability does not substantially or significantly decrease” when compared to cells grown in a standard medium without a disaccharide or a trisaccharide, means that cell viability does not decrease any more than about 15% compared to the control cultures (i.e. cells grown without a disaccharide or a trisaccharide).

As used herein, the phrase “without negative impact on efficiency on the production”, or equivalent thereof, means that the efficiency of production does not decrease any more than about 15% compared to the control cultures (i.e. cells grown without a disaccharide or a trisaccharide). In the context of the invention, as the efficiency of production run can be measured based on cell viability, viable cell density and/or harvest titre, it will be considered that there is no negative impact on the efficiency of production for instance if the VCD is at about −5% compared to the control or if the harvest titre is at or about −10% compared to the control.

The recombinant protein to be produced, in the context of the present invention as a whole, can be a therapeutic protein, an antibody or antigen binding fragment thereof, such as a human antibody or antigen-binding portion thereof, a humanized antibody or antigen-binding portion thereof, a chimeric antibody or antigen-binding portion thereof. Preferably, it is an antibody or antigen binding fragment thereof.

The methods of the present invention can be used to produce a protein, such as an antibody, having decreased amounts or levels of fucosyl residues and/or increased amounts or levels of mannosyl residues. Antibodies with such modified glycosylation profiles have been demonstrated to have an increased ADCC.

In the context of the invention as a whole, the trisaccharide compound, such as raffinose, is preferably present in a culture medium, or feed medium, or added to a culture medium, or feed medium, (as a supplement for instance) at a concentration of or of about 0.001 to 130 mM, even preferably at a concentration of or of about 0.01 to 100 mM, such as at concentration of or of about 0.001, 0.01, 0.05, 0.1, 0.5, 1, 2, 5, 10, 15, 20, 25, 30, 35, 40, 45, 50, 55, 60, 65, 70 or 100 mM (concentration of trisaccharide once in the culture medium, or feed medium, but before being in the culture system, i.e. before inoculation). For example, but not by way of limitation, by adjusting the concentration of a trisaccharide, while keeping the osmolality of the culture medium constant, the glycosylation profile as well as the efficiency of the production run(s) can be modulated. Alternatively, the trisaccharide can be added as a supplementary feed. In such a case, it will be added in similar starting concentration as above.

In the context of the invention as a whole, the disaccharide compound, such as sucrose, is preferably present in a culture medium, or a feed medium or added to a culture medium, or feed medium (as a supplement for instance) at a concentration of or of about 0.001 to 150 mM, even preferably at a concentration of or of about 0.01 to 130 mM, such as at concentration of or of about 0.001, 0.01, 0.05, 0.1, 0.5, 1, 2, 5, 10, 15, 20, 25, 30, 35, 40, 45, 50, 55, 60, 65, 70, 100 or 130 mM (concentration of trisaccharide once in the culture medium, or the feed medium, but before being in the culture system, i.e. before inoculation). For example, but not by way of limitation, by adjusting the concentration of a disaccharide, while keeping the osmolality of the culture medium constant, the glycosylation profile as well as the efficiency of the production run(s) can be modulated. Alternatively, the disaccharide can be added as a supplementary feed. In such a case, it will be added in similar starting concentration as above.

For the purposes of this invention, cell culture medium is a medium suitable for growth of animal cells, such as mammalian cells, in in vitro cell culture. Cell culture media formulations are well known in the art. Cell culture media may be supplemented with additional standard components such as amino acids, salts, sugars, vitamins, hormones, and growth factors, depending on the needs of the cells in culture. Preferably, the cell culture media are free of animal components; they can be serum-free and/or protein-free. Standard media have an osmolality of between 300 to 330 mOsm/kg, such as at or about 315 mOsm/kg. When the culture medium to be used comprise a disaccharide or a trisaccharide and should have an osmolality similar to the one of a standard medium, said culture medium is preferably a medium depleted in salt, such as in NaCl, MgCl2 and/or CaCl2), depending on the salts normally present in said medium. Once the disaccharide or trisaccharide is added at the needed concentration, the osmolality is controlled by introduction of at least one salt.

In certain embodiments of the present invention, the cell culture medium is supplemented with the disaccharide or the trisaccharide, for example, at the start of culture, and/or in a fed-batch or in a continuous manner. The addition of the disaccharide or trisaccharide supplement may be based on measured intermediate glycosylation profiles, or an measured intermediate efficiency of at least one production run.

In the context of the invention as a whole, the recombinant cell, preferably mammalian cell, is grown in a culture system such as a bioreactor. The bioreactor is inoculated with viable cells in a culture medium comprising or supplemented with a disaccharide, such as sucrose, or a trisaccharide, such as raffinose. Preferably the culture medium is serum-free and/or protein-free. Once inoculated into the production bioreactor the recombinant cells undergo an exponential growth phase. The growth phase can be maintained using a fed-batch process with bolus feeds of a feed medium optionally supplemented with said disaccharide or trisaccharide. Preferably the feed medium is serum-free and/or protein-free. These supplemental bolus feeds typically begin shortly after the cells are inoculated into the bioreactor, at a time when it is anticipated or determined that the cell culture needs feeding. For example, supplemental feeds can begin on or about day 3 or 4 of the culture or a day or two earlier or later. The culture may receive two, three, or more bolus feeds during the growth phase. Any one of these bolus feeds can optionally comprise or be supplemented with the disaccharide or the trisaccharide. The supplementation or the feed with the disaccharide or the trisaccharide can be done at the start of the culture, in fed-batch, and/or in continuous manner. The culture medium can comprise glucose or be supplemented by glucose. Said supplementation can be done at the start of the culture, in fed-batch, and/or in continuous manner.

The methods, compositions and uses according to the present invention may be used to improve the production of recombinant proteins in multistep culture processes. In a multiple stage process, cells are cultured in two or more distinct phases. For example cells are cultured first in one or more growth phases, under conditions improving cell proliferation and viability, then transferred to production phase(s), under conditions improving protein production. In a multistep culture process, some conditions may change from one step (or one phase) to the other: media composition, shift of pH, shift of temperature, etc. The growth phase can be performed at a temperature higher than in production phase. For example, the growth phase can be performed at a first temperature from about 35° C. to about 38° C., and then the temperature is shifted for the production phase to a second temperature from about 29° C. to about 37° C. The cell cultures can be maintained in production phase for days or even weeks before harvest.

In an embodiment of the present invention, the host cell is preferably a mammalian host cell (herein also refer to as a mammalian cell) including, but not limited to, HeLa, Cos, 3T3, myeloma cell lines (for instance NS0, SP2/0), and Chinese hamster ovary (CHO) cells. In a preferred embodiment, the host cell is Chinese Hamster Ovary (CHO) cells.

The cell lines (also referred to as “recombinant cells”) used in the invention are genetically engineered to express a protein of commercial or scientific interest. Methods and vectors for genetically engineering of cells and/or cell lines to express a polypeptide of interest are well known to those of skill in the art; for example, various techniques are illustrated in Ausubel et al. (1988, and updates) or Sambrook et al. (1989, and updates). The methods of the invention can be used to culture cells that express recombinant proteins of interest. The recombinant proteins are usually secreted into the culture medium from which they can be recovered. The recovered proteins can then be purified, or partially purified using known processes and products available from commercial vendors. The purified proteins can then be formulated as pharmaceutical compositions. Suitable formulations for pharmaceutical compositions include those described in Remington's Pharmaceutical Sciences, 1995.

The recombinant protein with a modulated glycosylation profile, for example an antibody or antigen-binding fragment thereof, with a decreased fucosylation level or amount and/or an increased mannosylation level or amount, produced by a method of the present invention may be used to treat any disorder in a subject for which the therapeutic protein (such as an antibody or an antigen binding fragment thereof) comprised in the composition is appropriate for treating.

In a further aspect, also disclosed are pharmaceutical compositions comprising the recombinant protein with a modulated glycosylation profile produced by the methods of the invention and a pharmaceutically acceptable carrier. The recombinant protein is preferably a therapeutic protein, and can be an antibody or antigen binding fragment thereof, such as a human antibody or antigen-binding portion thereof, a humanized antibody or antigen-binding portion thereof, a chimeric antibody or antigen-binding portion thereof. Preferably, it is an antibody or antigen binding fragment thereof, with a decreased fucosylation level or amount and/or an increased mannosylation level or amount.

In certain embodiments, the pharmaceutical compositions of the invention comprising a recombinant protein with a modulated glycosylation profile may be formulated with a pharmaceutically acceptable carrier as pharmaceutical (therapeutic) compositions, and may be administered by a variety of methods known in the art (see for instance Remington's Pharmaceutical Sciences, 1995). Such pharmaceutical compositions may comprise any one of salts, buffering agents, surfactants, solubilizers, polyols, amino acids, preservatives, compatible carriers, optionally other therapeutic agents, and combinations thereof. The pharmaceutical compositions of the invention comprising a recombinant protein with a modulated glycosylation profile, are present in a form known in the art and acceptable for therapeutic uses, such as liquid formulation, or lyophilized formulation. Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features.

The present disclosure is therefore to be considered as in all aspects illustrated and not restrictive, the scope of the invention being indicated by the appended Claims, and all changes which come within the meaning and range of equivalency are intended to be embraced therein.

The foregoing description will be more fully understood with reference to the following examples. Such Examples, are, however, exemplary of methods of practising the present invention and are not intended to limit the scope of the invention.

### EXAMPLES

### Material and Methods

### I. Cells, Cell Expansion and Cell Growth

### 1) Cells

Assays were performed with two CHO cell lines:
CHO-S cells expressing IgG1 mAb1, herein “Cells mAb1” or “mAb1 cells”. “mAb1” is a fully human monoclonal antibody directed against a soluble protein. Its isoelectric point (pI) is about 8.20-8.30.
CHO-K1 cells expressing IgG1 mAb2, herein “Cells mAb2” or “mAb2 cells”. “mAb2” is a humanized monoclonal antibody directed against a receptor found on the cell membrane. Its isoelectric point (pI) is about 9.30.

### 2) Cell Expansion

Cell expansion was performed in tubes in a medium suitable for cell expansion. Assays in fed-batch started after at least one week expansion.

### 3) Inoculation

Deepwell plates: Cells expressing mAb2 were inoculated at 0.2×10⁶ cells per millilitre (mL), whereas cells expressing mAb1 were inoculated at 0.3×10⁶ cells per mL.

Spintubes: Cells expressing both mAb1 and mAb2 were inoculated at 0.3×10⁶ cells per mL.

### 4) Fed-Batch

All assays were performed in fed-batch culture.

A serum-free chemically defined culture medium was used. It was used as it is (o be adapted), or it was supplemented with D-(+)-Raffinose pentahydrate (Sigma-Aldrich, 83400-25G) at different concentrations (0-45 mM). The culture medium was fed, on a regular basis, with a chemically defined feed medium, as well as with glucose in order to keep said glucose level in the range of >0 to about 8 g/L.

The cultures were performed:
In deepwell plates with a working volume of 450 μL. They were incubated at 36.5° C., 5% de CO₂, 90% humidity and shaken at 320 rpm. Each of the fed-batch culture lasted 14 days.
In Spin Tubes (ST) with a working volume of 30 mL (with as permable lid). They were inoculated with a cell density of 0.3*10⁶ cells/mL and maintained at 36.5° C., 320 rpm, 5% CO2 and 90% humidity for 14 days.

### II. Analytical Methods

Viable cell density and viability were measured with the Guava easyCyte® flow cytometer.

Antibody titers were measured with the fortéBIO Octet®.

Glycosylation profiles were established by capillary gel electrophoresis with laser-induced fluorescence (CGE-LIF). Groups of glycans were defined as thereafter in Table 2.

### Example 1—Effect of Addition of a Disaccharide or Trisaccharide while Keeping the Osmolality Constant (in Deep-Well Plates; Experimental Approach 1)

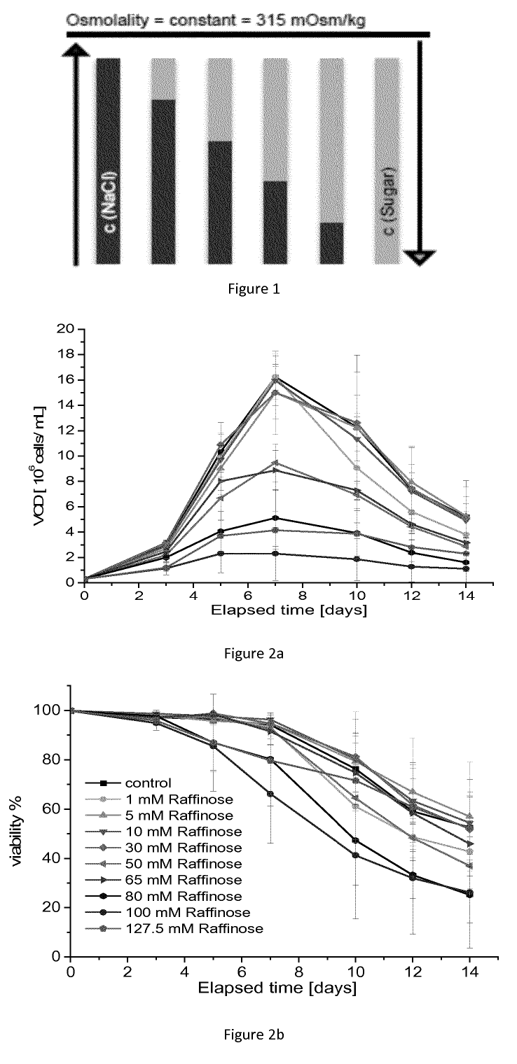

Experiment was performed to check whether high osmolality or high sugar concentration have an influence on the viability of the cells, VCD as well as on amount of High Mannose (HM) species. A chemically defined proprietary medium with lower osmolality (PM-200) compared to standard media, was used to vary sugar concentrations from 1-150 mM (green) while maintaining the osmolality of standard media (315 mOsm/kg) via supplementation with NaCl (blue), as illustrated in FIG. 1. Standard media and PM-200 differ in the composition, so the missing amounts of raw material were added (except NaCl). As sugars raffinose (a trisaccharide) and sucrose (a disaccharide) were chosen. Stock solutions (raffinose 22 mM, raffinose 220 mM, sucrose 50 mM, sucrose 1 M and NaCl 1 M) were prepared and added to the media before inoculation.

Table 3 summarizes the different conditions of experimental approach 1 and 2. Stock solutions were prepared with media to prevent dilution. The given concentrations equal the concentrations in media before incoculation. CHO-S cells (=mAb1 cells) expressing mAb1 were expanded for 49 days, CHO-K1 (=mAb2 cells) expressing mAb2 were expanded for 28 days.

TABLE 3
experimental approach 1 and 2: 1: constant osmolality (315 mOsm/kg) but increasing
sugar concentration (0-127.5 mM), 2: constant sugar concentration (0 or 30 mM)
with increasing osmolality (300-375 mOsm/kg); n = 5-6

| Experimental approach 1 | | | Experimental approach 2 | | |
|---|---|---|---|---|---|
| | | Concentration of | | | Concentration |
| | Osmolality | raffinose/ | | Osmolality | of raffinose |
| Condition | [mOsm/kg] | sucrose [mM] | Condition | [mOsm/kg] | [mM] |
| 1 | 315 | 0 | 1 | 300 | 0 |
| 2 | 315 | 1 | 2 | 300 | 30 |
| 3 | 315 | 5 | 3 | 315 | 0 |
| 4 | 315 | 10 | 4 | 315 | 30 |
| 5 | 315 | 30 | 5 | 375 | 0 |
| 6 | 315 | 50 | 6 | 375 | 30 |
| 7 | 315 | 65 | | | |
| 8 | 315 | 80 | | | |
| 9 | 315 | 100 | | | |
| 10 | 315 | 127.5 | | | |

Results—Addition of Raffinose on mAb1 Cells in Culture:

The impact of constant osmolality (315 mOsm/kg) but increasing concentration of raffinose on mAb1 cell growth is illustrated in FIG. 2. With increasing sugar concentration, cell growth was inhibited. The maximum cell concentration of 16.2±1.0*10⁶ cells/mL was reached by the control and the concentration of 1 mM raffinose while with 100 mM raffinose only 2.3±2.1*10⁶ cells/mL were reached. From working day 07, cell viability was lower with high raffinose concentration. Highest viability was at 5 mM raffinose (about 57%), whereas the control obtained about 53% on working day 14

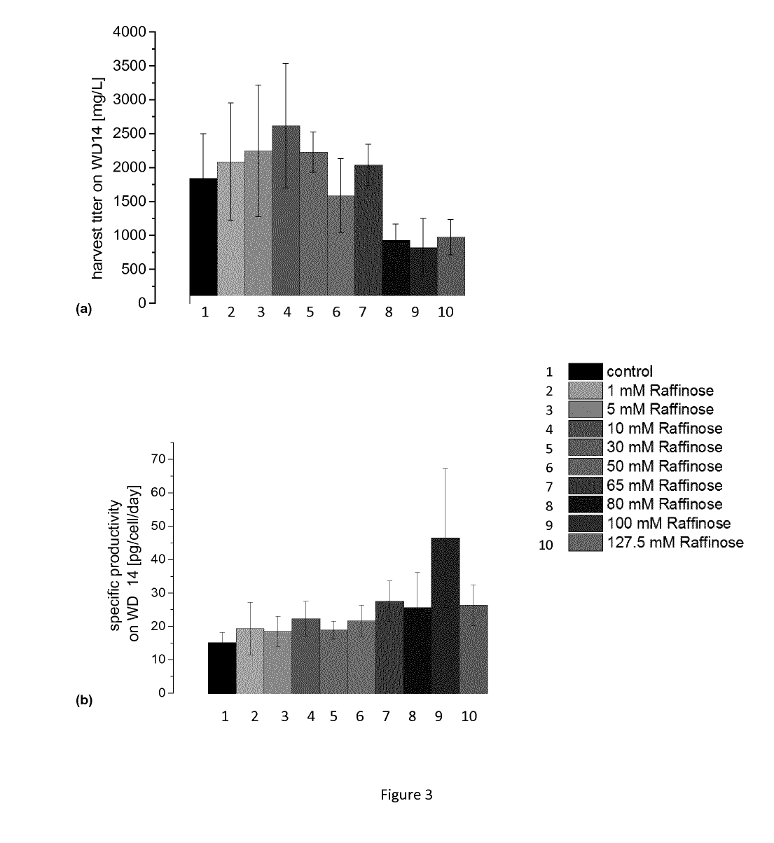

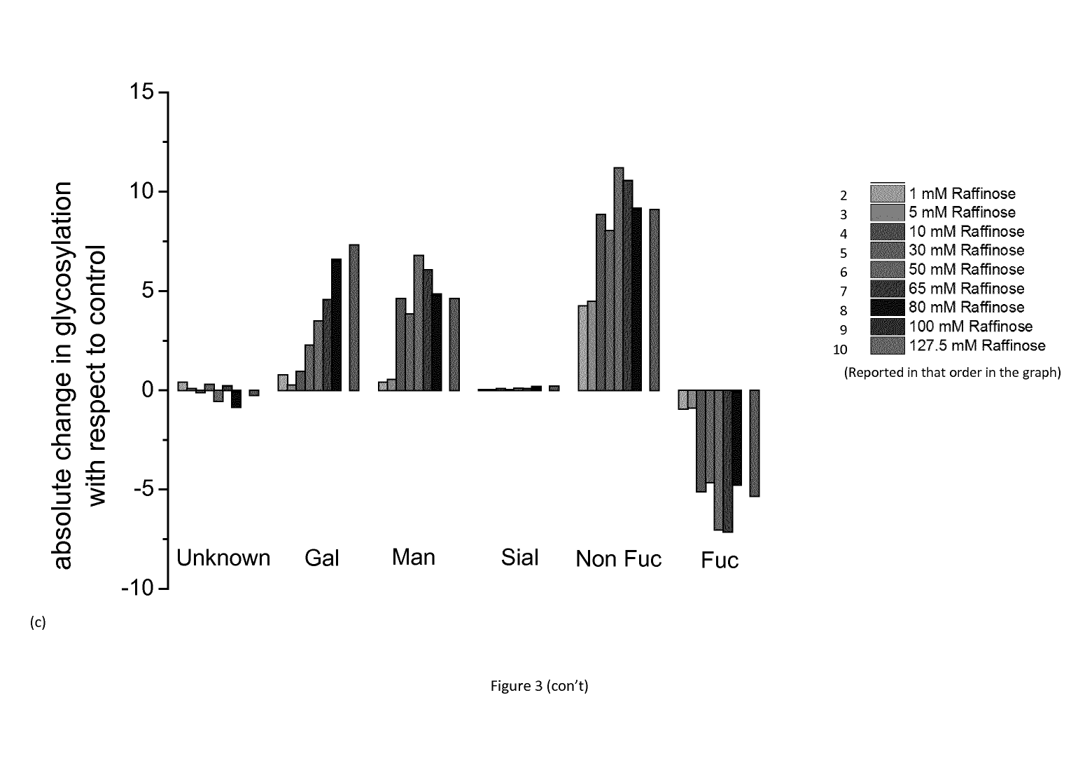

FIG. 3a shows the absolute harvest titer on working day 14 of CHO-S cells with constant osmolality (315 mOsm/kg) and supplementation of raffinose in media. Conditions with high concentrations of raffinose (80-127.5 mM) resulted in titers of 825-975 mg/L, whereas the control amounted to about 1850 mg/L. The condition with 10 mM raffinose achieved the highest titer of about 2650 mg/L. All together, conditions with 1-65 mM raffinose obtained a higher product titer than control, data not shown. Specific productivity [pg/cell/day] is shown in FIG. 3b. With increasing raffinose concentration, specific productivity increased, likewise. Highest specific productivity (about 45 pg/cell/day) was at 100 mM raffinose.

Supplementation of 50 mM raffinose achieved the highest percentage increase (6.8%) of HM species amount FIG. 3c). With increasing sugar concentration an increase of galactosylated, HM and non-fucosylated species was observed as well as a decrease of fucosylated species.

In summary, example 1 shows that increasing raffinose concentration at constant osmolality affects growth rate, viability, antibody production as well as the glycosylation profile of mAb1. Cultures with 1-30 mM raffinose show the highest VCD, viability and antibody concentration on working day 14. At 50 mM raffinose the highest increase of HM (6.8%) was observed. This indicates that high sugar concentration decreases cell growth as well as increases specific antibody production and results in a significant change of the glycosylation profile of mAb1.

Results—Addition of Raffinose on mAb2 Cells in Culture:

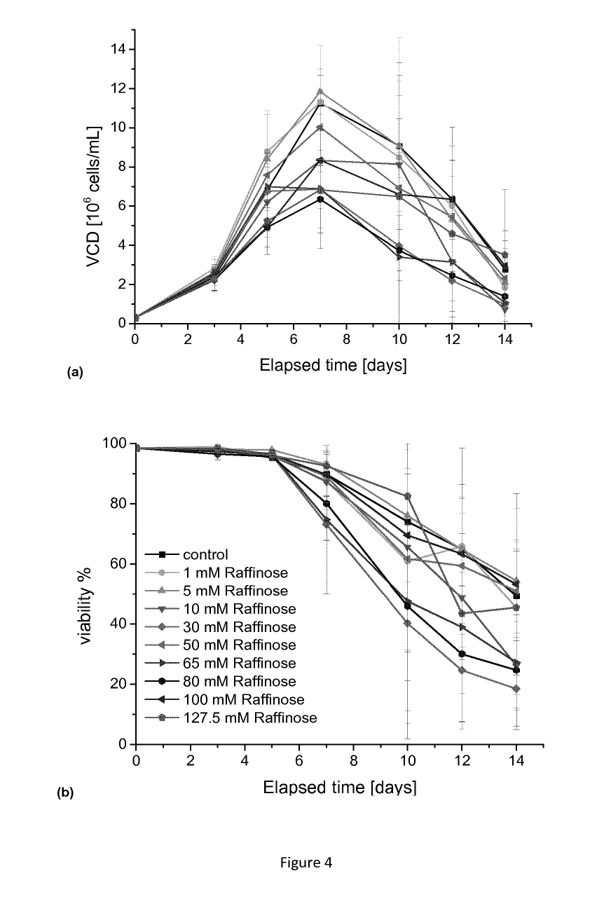

The impact of constant osmolality and increasing concentration of raffinose on mAb2 cell growth and viability of cells are highlighted in FIG. 4.

FIG. 4a shows that with increasing sugar concentration, cell growth of mAb2 cells was reduced. At 5 mM of raffinose the maximum cell concentration of 11.85±1.1*10⁶ cells/mL was obtained, while the control reached a maximum VCD of 11.3±1.4*10⁶ cells/mL. The lowest VCD was obtained by the condition with additional 80 mM raffinose (6.4±1.7*10⁶ cells/mL). From day 7 on, viability of conditions with increasing sugar concentration decreased (FIG. 4b). The viability of cultures with high concentration of raffinose was significantly lower than control at working day 14.

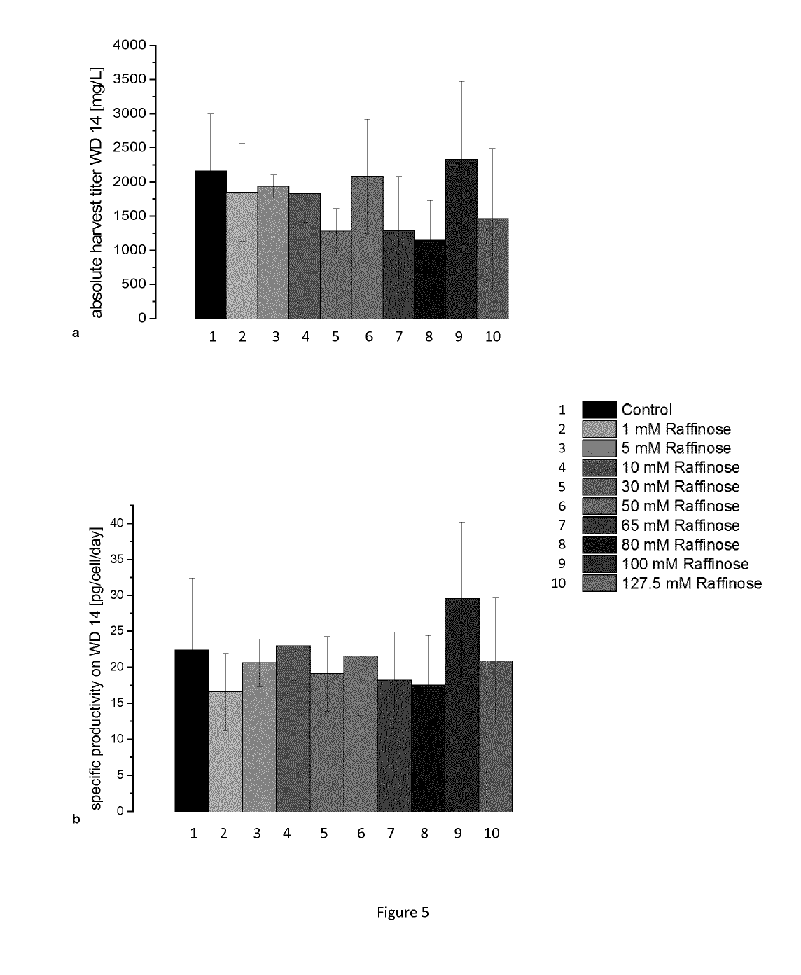

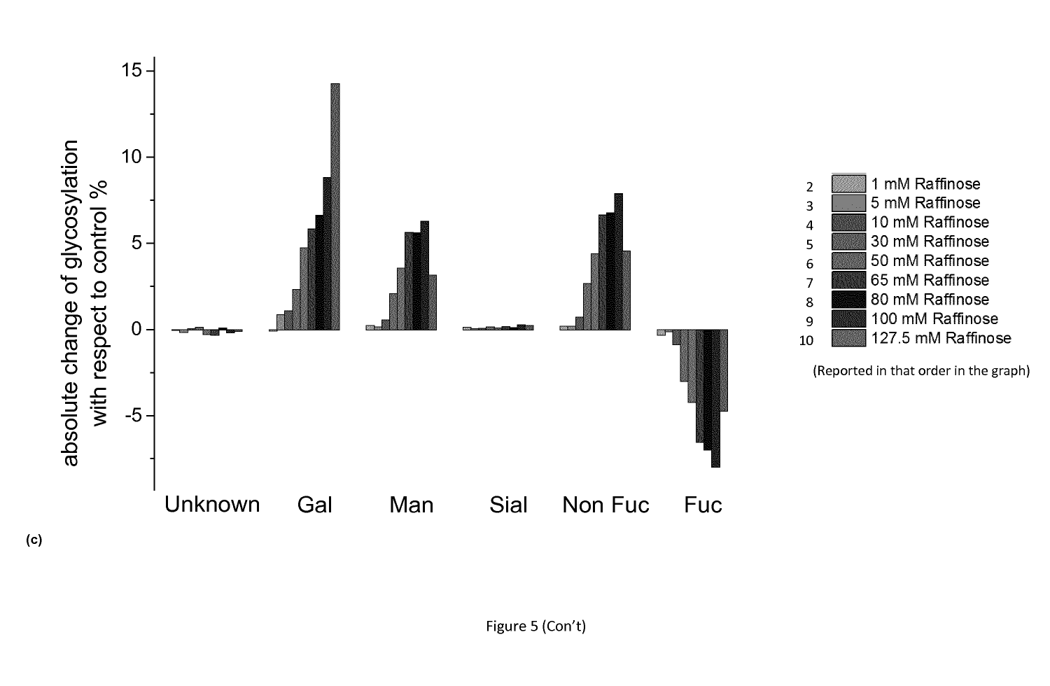

FIG. 5a shows the relative harvest titer on working day 14 of mAb2 cells with supplementation of raffinose in media. With increasing raffinose concentration the relative harvest titer decreased, except for conditions with 50 mM and 100 mM raffinose, which gained the highest antibody concentration. The condition with 100 mM raffinose obtained about 2350 mg/L, whereas the control produced about 2150 mg/L of mAb2 on working day 14.

Specific productivity is shown in FIG. 5b. Compared to the control, there were no changes in the productivity of mAb2 with increased raffinose, except for the condition with 100 mM raffinose. This condition obtained the highest productivity (about 30 pg/cell/day), while the control reached about 22 pg/cell/day.

With higher sugar concentration an increase of galactosylated, HM and non-fucosylated species was observed as well as a decrease of fucosylated species (FIG. 5c). Supplementation of 100 mM raffinose increased the HM species by 6.3%.

In summary, as with mAb1 cells, increasing raffinose concentration at constant osmolality affects growth rate, viability and absolute titer on working day 14. The figure with absolute change in glycosylation (FIG. 24c) shows similar tendencies compared to the experimental approach with mAb1 cells. Galactosylated glycoforms increased with increasing sugar concentration but the increase was higher with mAb2 cells. Non-fucosylated glycoforms increased, fucosylated glycoforms decreased with increasing raffinose concentration. The highest increase of HM species of cultures with mAb2 cells were obtained by 100 mM (6.3%).

Results: Addition of Sucrose on mAb1 Cells in Culture

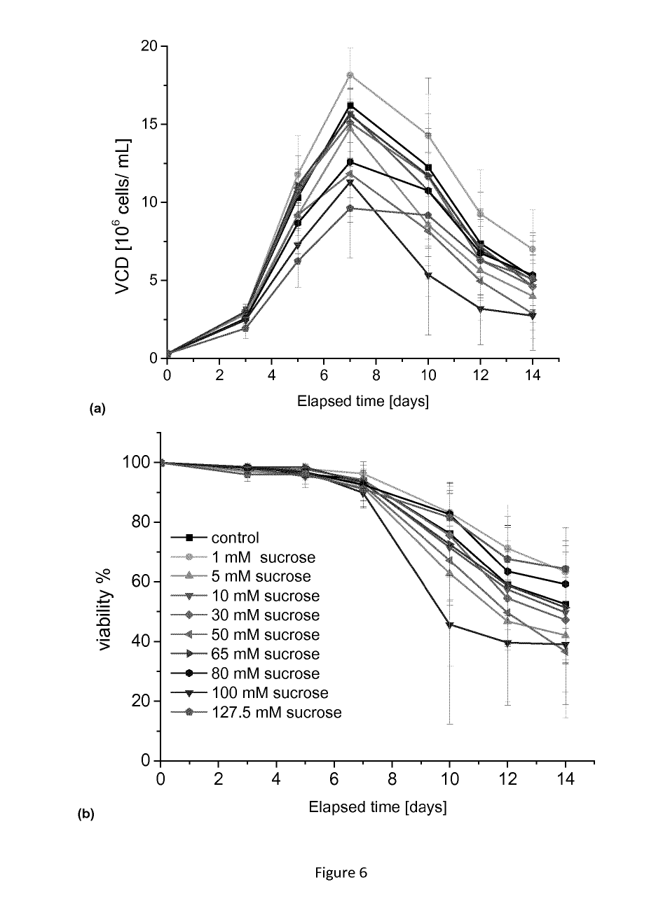

The results of the experimental approach with constant osmolality but increasing concentration of sucrose on mAb1 cell growth are illustrated in FIG. 6. Likewise, with increasing sugar concentration, cell growth was inhibited. The maximum cell concentration of 18.2±1.7*10⁶ cells/mL was reached at 1 mM sucrose, while the condition at the maximum tested sucrose concentration (127 mM) sucrose reached 9.6±3.2*10⁶ cells/mL.

After working day 7, viability was lower with increasing sucrose concentration than the control except for the condition with 1 mM, 80 mM, and 127.5 mM sucrose. Highest viability was obtained at 1 mM and 127.5 mM sucrose (about 635% and 64%), while control obtained about 53%.

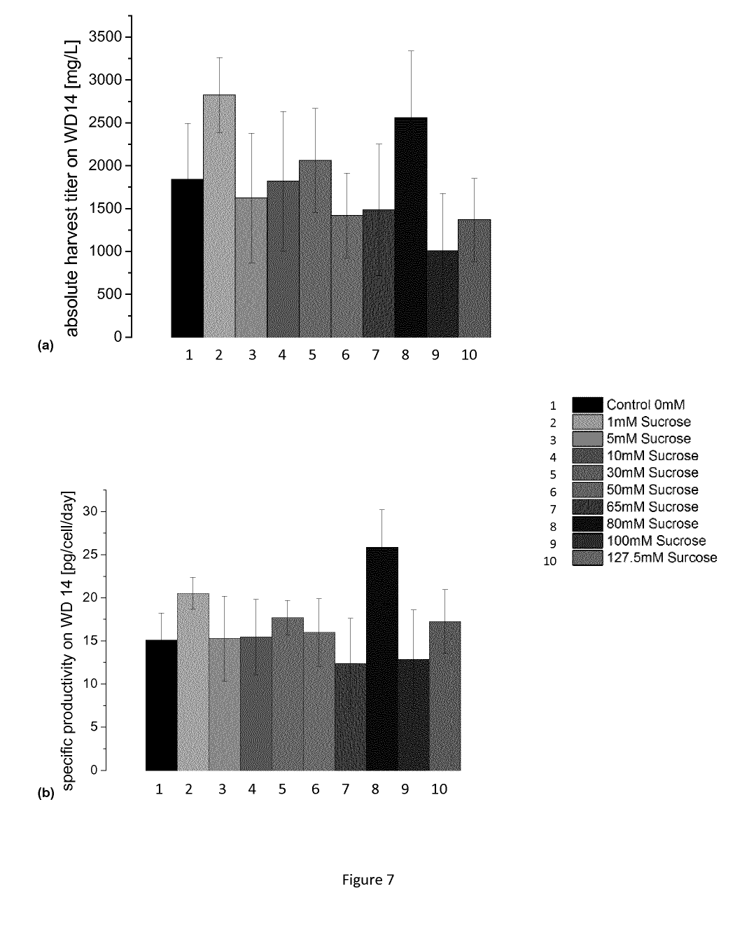

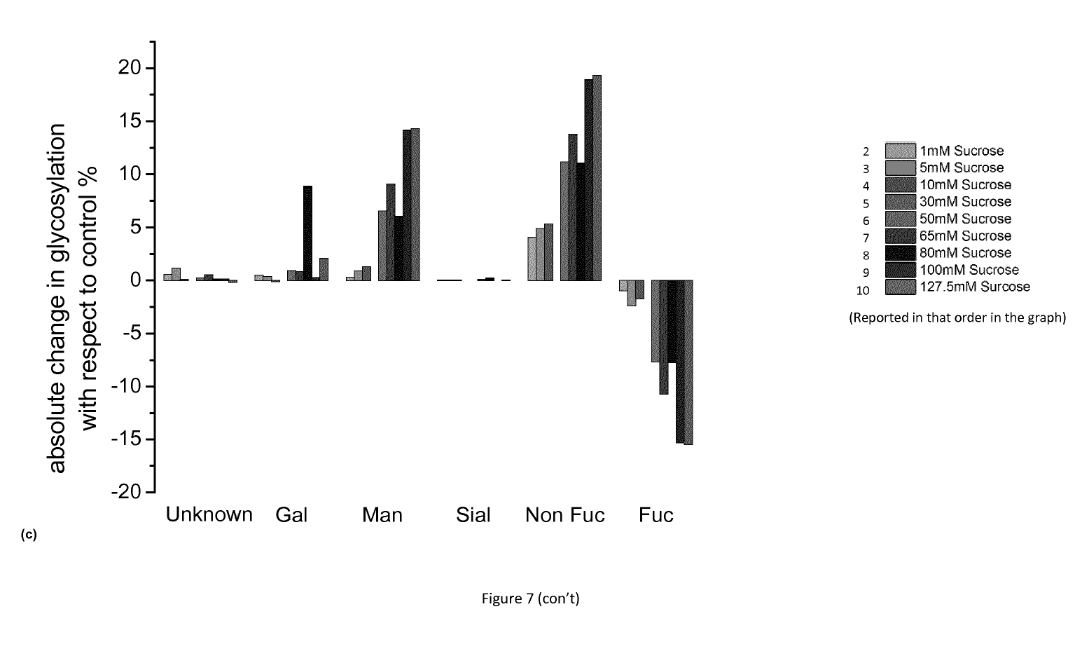

With increasing concentration of sucrose the absolute harvest titer decreased except from conditions with 1 mM and 80 mM supplementation. Those conditions resulted in the highest absolute harvest titer on working day 14 (about 2800 mg/L and 2550 mg/L), whereas the titer in the control was about 1850 mg/L (FIG. 7a).

With increasing sucrose concentration there was no change in the specific productivity on working day 14 (FIG. 7b), except for the condition with 1 mM and 80 mM sucrose. The highest productivity was obtained at 80 mM sucrose (about 26 pg/cell/day).

Supplementation of 100 mM and 127.5 mM sucrose increased the HM species by 14.2% and 14.3% (FIG. 7c). With greater sugar concentration an increase of galactosylated, HM and non-fucosylated species was observed as well as a decrease of fucosylated species.

In summary, the highest VCD is obtained by the control and the following conditions: 1 mM, 5 mM, 10 mM, 30 mM and 65 mM sucrose. Conditions with 1 mM, 80 mM and 100 mM sucrose displayed the highest viability on working day 14. For the conditions with higher sucrose concentration, higher viability was probably due to lower cell density during the cultivation. The best glycosylation profile was obtained by the condition with the highest sucrose concentration (100 mM and 127.5 mM) with an increase of 14.2% and 14.3% HM.

This confirms the assumption from the experiment above (mAb1 cells in DWP—supplementation of raffinose) that high sugar concentration decrease cell growth and increase specific productivity.

Results—Addition of Sucrose on mAb2 Cells in Culture:

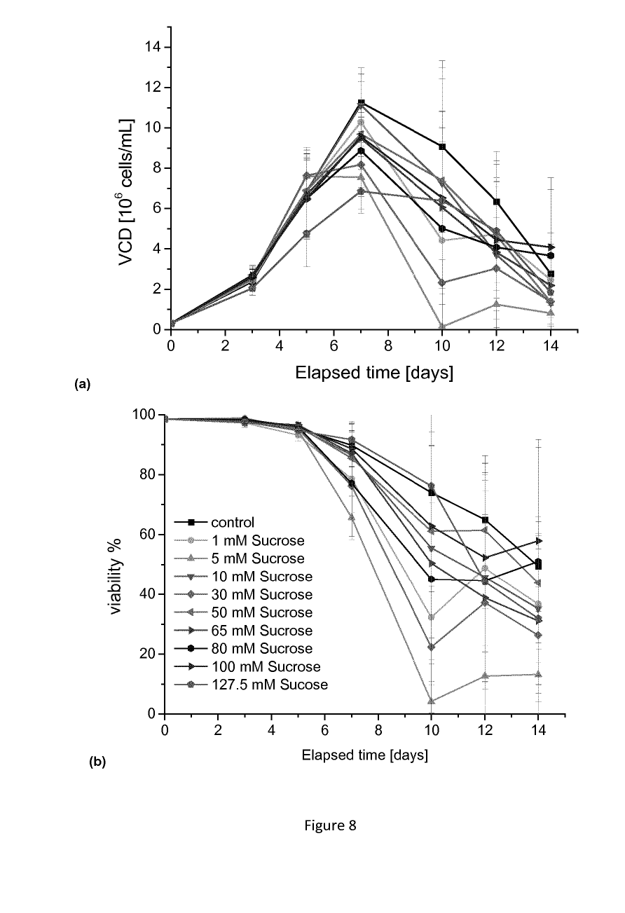

FIG. 8 depicts VCD and viability from mAb2 cells of the experimental approach with constant osmolality (315 mOsm/kg) and increasing sucrose concentration.

The maximum cell concentration of 11.3±1.4*10⁶ cells/mL obtained the control, while the condition of 10 mM sucrose reached 11.1±1.6*10⁶ cells/mL VCD. The lowest VCD obtained the condition with 127.5 mM sucrose (6.9±1.1*10⁶ cells/mL). From working day 07, decreased viability with increasing sugar was observed.

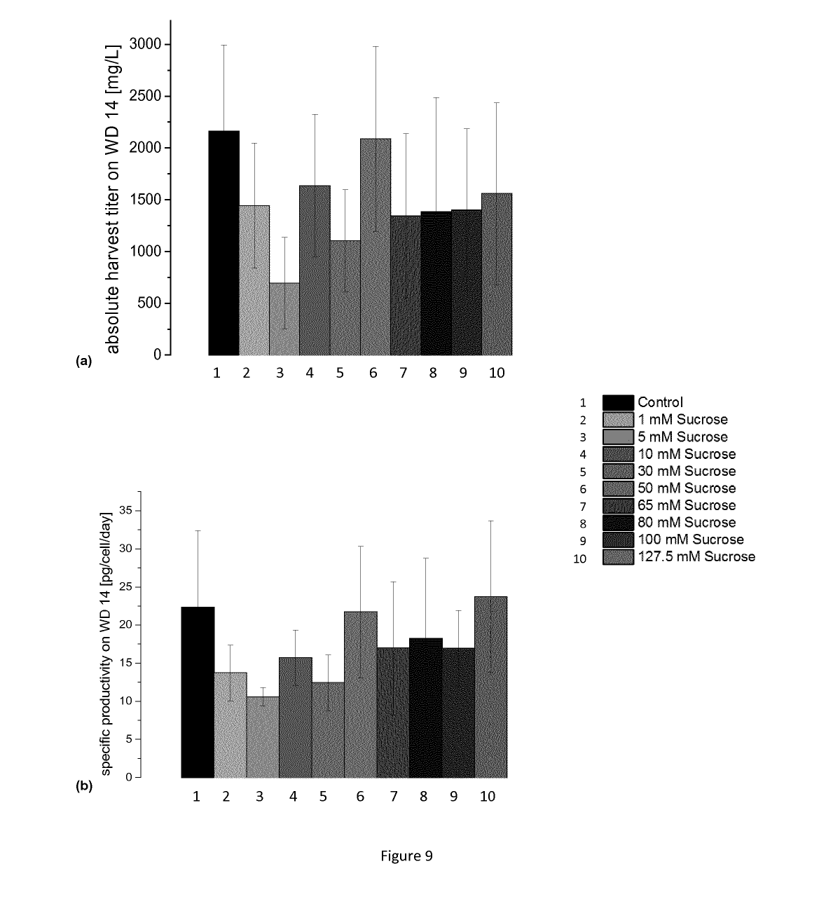

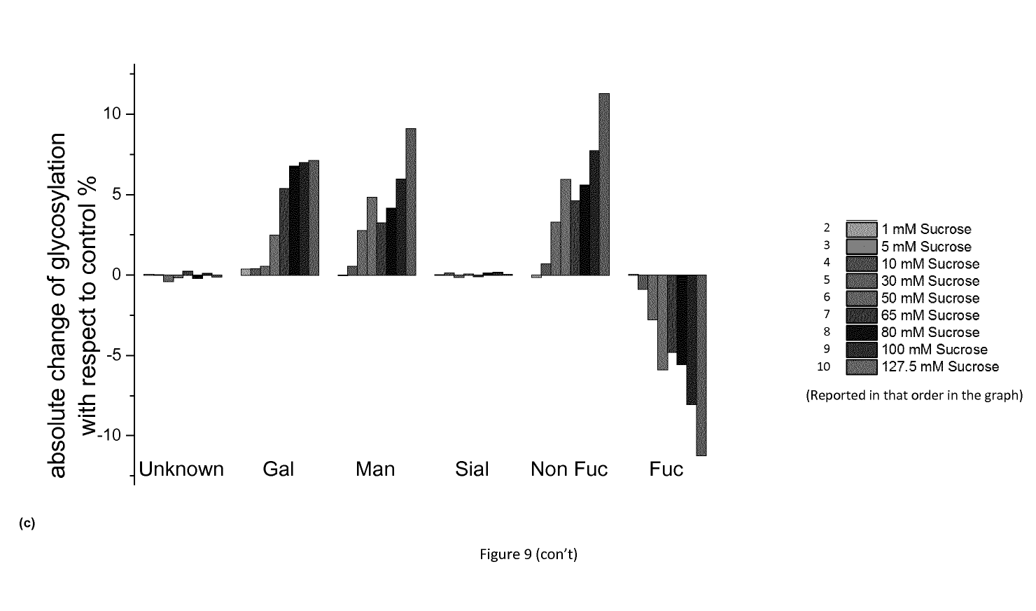

FIG. 9a shows the absolute harvest titer on working day 14 of mAb2 cells with supplementation of raffinose in media. Supplementation of sucrose resulted in a decrease of the production of antibodies with respect to control. The highest absolute titer of about 2150 mg/L was obtained by the control and by the condition with 50 mM sucrose (about 2100 mg/L), whereas the lowest concentration was obtained by 5 mM sucrose (about 700 mg/L).

Compared to the control (about 22 pg/cell/day), specific productivity was lower except for the conditions with 50 mM (about 21 pg/cell/day) and 127.5 mM sucrose (about 24 pg/cell/day) on working day 14 (FIG. 9b).

FIG. 9c depicts the change of the glycosylation profile with respect to the control. Supplementation of 127.5 mM sucrose increased the HM species by 9.1%; 100 mM sucrose increased the amount HM species by 6.0%. With greater sugar concentration an increase of galactosylated, HM and non-fucosylated species was observed as well as a decrease of fucosylated species.

In summary, in the course of cultivation, the viability of the sucrose supplemented cultures was lower than the viability of the control. After working day 7, the VCD significantly decreased because of very likely too low glucose levels over the weekend or limitation of other media components. This assumption is confirmed by the increase of VCD on working day 12 after glucose and main feed was fed again. Absolute harvest titer and specific productivity on working day 14 of cultures with supplemented sucrose was significant lower than the titer of control. Compared with mAb1 cells, the increase of HM and non-fucosylated glycoforms was lower. Likewise, the decrease of fucosylated glycans was lower, but an increase of galactosylation was obtained only with mAb2 cells.

### Example 2: Effect of Addition of a Disaccharide or Trisaccharide while Keeping the Osmolality Constant (in Spin Tubes; Experimental Approach 1)

To verify the results of example 1, the experimental approach 1 was repeated in 50 mL Spin Tubes and with cells mAb1 (27 days expansion) with the conditions given in Table 4. Again, the given osmolality and concentrations equal the conditions before the inoculation.

TABLE 4
four experimental conditions with constant osmolality (315 nnOsm/kg)
but increasing concentration of raffinose (0-100 mM); n = 2

| Condition | Osmolality [mOsm/kg] | Concentration of raffinose [mM] |
|---|---|---|
| 1 | 315 | 0 |
| 2 | 315 | 10 |
| 3 | 315 | 50 |
| 4 | 315 | 100 |

Results—Addition of Raffinose on mAb1 Cells in Culture:

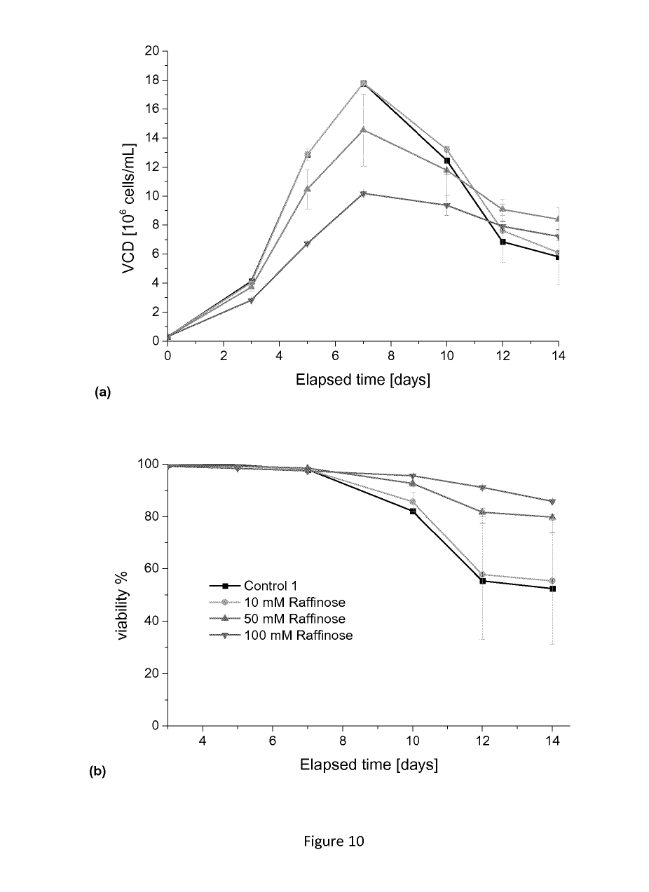

A second experiment with constant osmolality (315 mOsm/kg) and increasing raffinose concentration was performed in Spin Tubes with a working volume of 30 mL and mAb1 cells. FIG. 10a depicts VCD and viability from mAb1 cells of the experimental approach with constant osmolality and increasing raffinose concentration in Spin Tubes. The maximum cell concentration of 17.8±0.2*10⁶ cells/mL was reached in the control and at 10 mM raffinose (17.8±0.2*10⁶ cells/mL). The lowest VCD was obtained at 100 mM raffinose (10.2±0.2*10⁶ cells/mL), as illustrated in FIG. 10b. But this condition showed the best viability at the end of cultivation (about 86%), while the worst viability was observed in the control (about 52%).

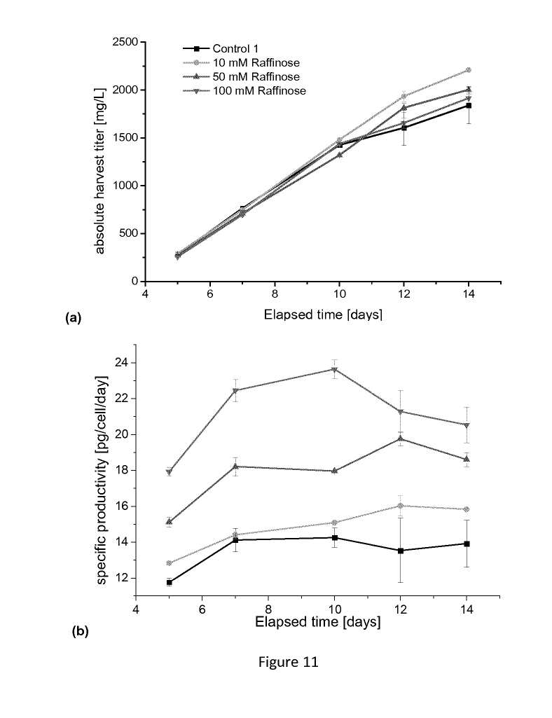

The highest concentration of antibodies on working day 14 was achieved by the condition with 10 mM raffinose (about 2200 mg/L), while the control reached about 1840 mg/L and therefore the lowest titer, as illustrated in FIG. 11a. The conditions with supplementation of raffinose showed all a better productivity than the control, see FIG. 11b. While control had a specific productivity of about 14 pg/cell/days, the highest productivity per cell per day (PCD) obtained the condition with 100 mM raffinose (peak at about 23 pg/cell/day) and achieved an increase of productivity (about 64%). The PCD of cultures with 50 mM and 100 mM raffinose showed a steeper slope than condition with 10 mM raffinose and control.

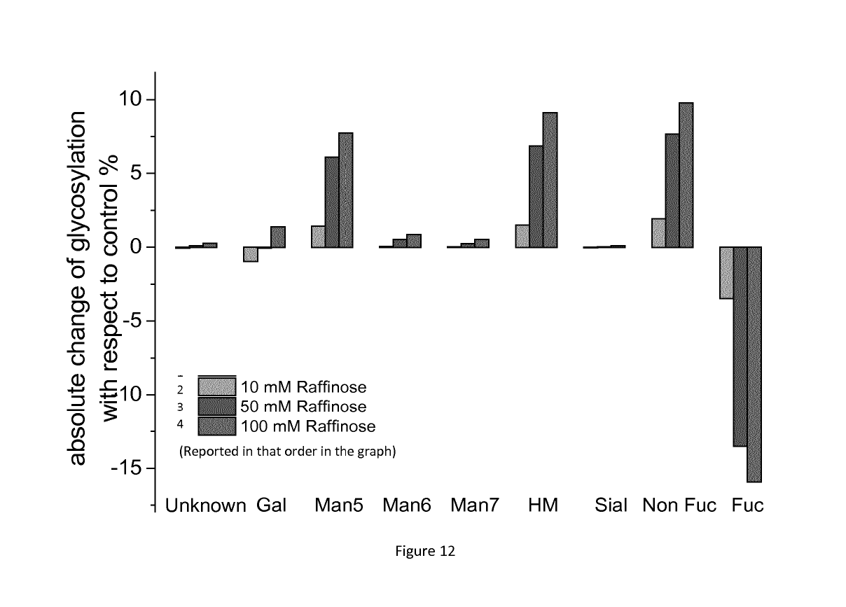

Supplementation of raffinose allowed an increase of the amount of Man5 and non-fucosylated as well as a decrease of fucosylated glycoforms (FIG. 12). An increase of Man5 by 7.7% and decrease of fucosylated glycans by 15.9% was obtained at 100 mM raffinose. The absolute change of unknown, galactosylated and sialylated glycoforms was not affected.

With increasing raffinose concentration, the amount of alkaline isoforms decreased, while acid isoforms were increased as well as the amount of aggregates (data not shown).

The absolute harvest titer from cultures with supplemented raffinose was higher than the control (FIG. 11a). Compared with the same experimental approach in DWP, the cultures demonstrated similar behavior, but with 1-65 mM raffinose, only. Although the VCD of condition 100 mM raffinose was the lowest, antibody concentration at working day 14 stays in the same range as control, resulting in a higher productivity than control. One possible explanation could be that even more antibodies can be produced by larger cell diameter.

Increasing raffinose concentration at constant osmolality results in an increase of HM and non-fucosylated and decrease of fucosylated glycoforms. Sialylated and galactosylated glycoforms were not affected.

### Example 3: Effect of Addition of a Disaccharide or Trisaccharide while Varying the Osmolality (in Deep-Well Plates; Experimental Approach 2)

The second experimental approach was performed with increasing osmolality and constant sugar concentrations (see table 3 and methods in example 1).

Results—Addition of Raffinose on mAb2 Cells in Culture:

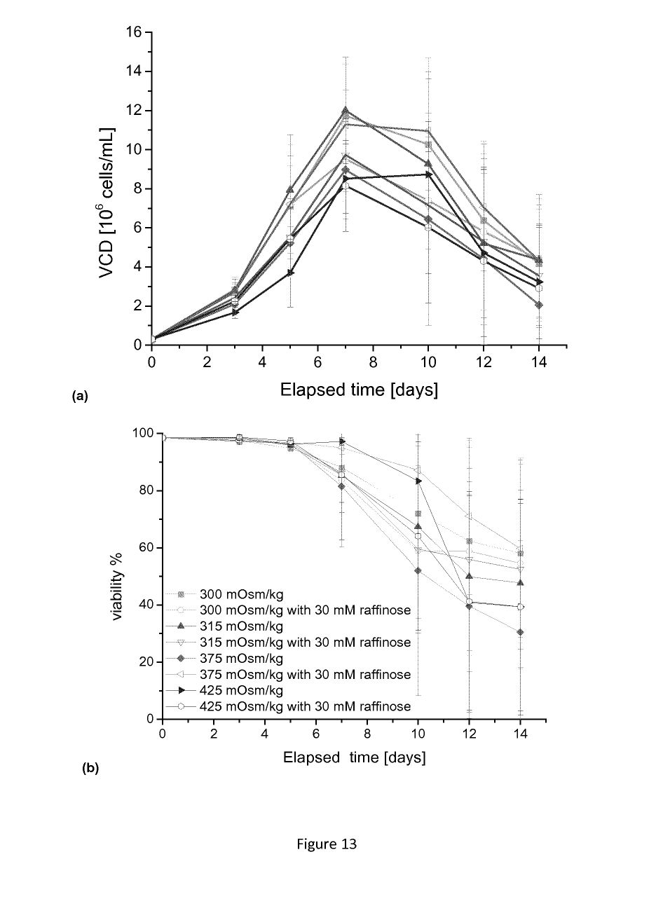

FIG. 13 shows VCD and viability of the cultures. Cultures with additional raffinose are labeled with “30 mM raffinose”. Control obtained the highest VCD of 11.7±2.7*10⁶ cells/mL. Increase of osmolality resulted in a decrease of maximum cell density. Additional supplementation of raffinose showed no correlation with decreasing VCD. The lowest VCD was obtained at 425 mOsm/kg with 30 mM raffinose (8.2±2.3*10⁶ cells/mL), see FIG. 13a. At the end of the experiment, the cultures with 375 mOsm/kg and 30 mM raffinose (about 59%) and 300 mOsm/kg (about 58%) exhibited the highest viability (FIG. 13b). Viability decreased with increasing osmolality, except for 375 mOsm/kg with 30 mM raffinose.

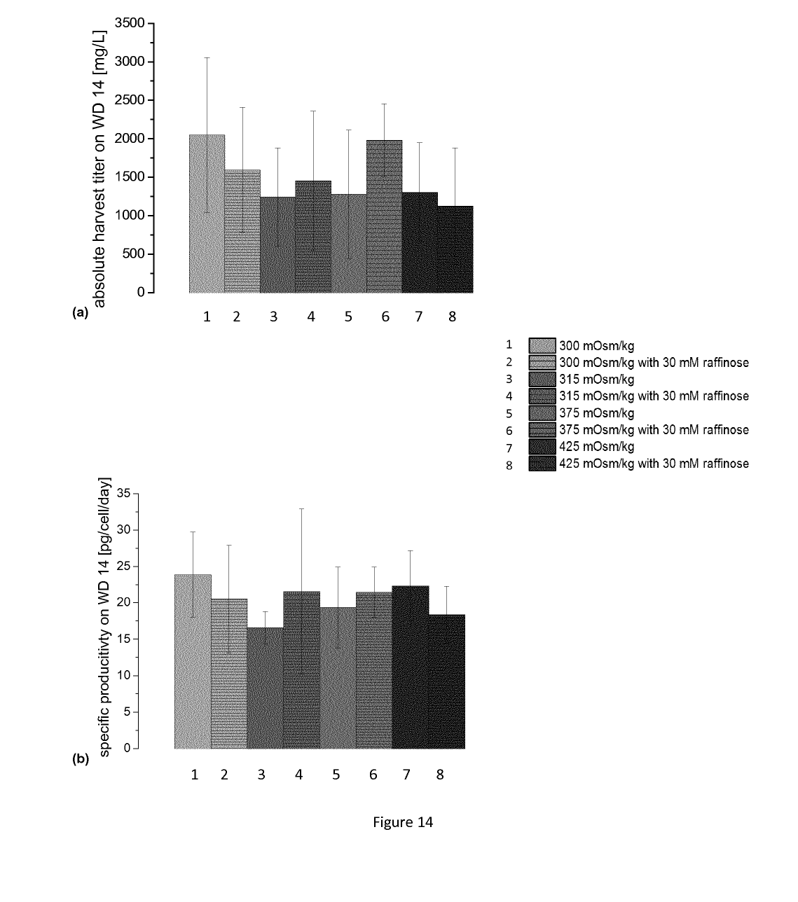

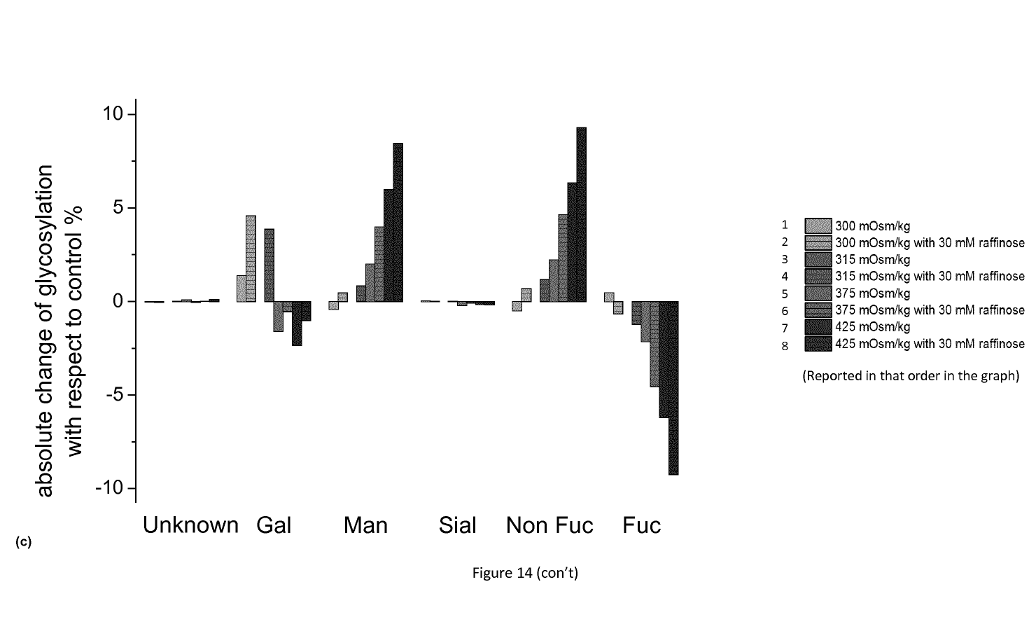

FIG. 14a depicts the absolute harvest titer on working day 14. Highest concentration of antibody was produced by control (about 2050 mg/L), whereas the condition with 425 mOsm/kg and supplementation of 30 mM raffinose only obtained about 1150 mg/L. Hence, with increasing osmolality, the absolute harvest titer decreased. Specific productivity (FIG. 14b) stayed in the range of about 18 pg/cell/day until about 24 pg/cell/day.

The change of the glycosylation profile with respect to control (315 mOsm/kg) can be seen in FIG. 14c. With increasing osmolality, the amount of fucosylated glycoforms decreased, while the non-fucosylated and galactosylated glycoforms are increased. An increase of HM species is observed, likewise. The condition 425 mOsm/kg with 30 mM raffinose achieved the highest increase 8.5%. The increase/decrease of each glycoform is even higher, when raffinose was added.

In summary, high osmolality and additional raffinose seem to inhibit cell growth, which may be explained by the downregulation of tubulin. In comparison to the experimental approach 1 (see examples 2 and 3), viability of conditions with high osmolality remain the same as viability of conditions with high sugar concentrations. There was no increase of antibody concentration on working day 14 compared to control, but similar specific productivity on working day 14. With increasing osmolality, the amount of HM species in all conditions increased.

### OVERALL CONCLUSION

Examples 1 and 2 underline that the addition of raffinose or sucrose, in a cell culture medium, at constant osmolality were able to affect growth rate, viability as well as the glycosylation profile of mAb1 and mAb2. For mAb1 an increase of HM by 7% or 14% were obtained when respectively raffinose or sucrose were added. For mAb2 an increase of HM by 6% or 9% were obtained when respectively raffinose or sucrose were added. Specific productivity was not affected by supplementation of sugar. It was thus shown that high disaccharide or trisaccharide concentrations decrease cell growth and increase specific productivity. Similar results were obtained both in DWP and in Spin Tubes.

The results presented here show that it is possible to control the efficiency of production runs as well as to control the abundance of HM species by supplementation of compounds like disaccharide (e.g. sucrose) or or trisaccharide (e.g. raffinose), while acting on the osmolality, preferably keeping it constant compared to a standard medium.

Based on the results presented in example 3, it is hypothesized that not only high sugar concentration but also high osmolality decreases cell growth and increases specific productivity.

The present invention surprisingly shows that it is possible to modulate the efficiency of at least one production runs and/or to modulate the glycosylation profile of proteins, such as antibodies, by controlling the concentrations in disaccharide or trisaccharide and osmolality of the culture medium. It is thus possible to adapt the culture conditions to specific goals in term of quantity and/or quality.

The skilled person will understand from the results of examples 1 to 3 that he can use a disaccharide (such as sucrose) or a trisaccharide (such as raffinose), while keeping the osmolality of the culture medium constant compared to a standard medium, for modulating the efficiency of at least one production runs and/or the glycosylation profile of any antibodies and any proteins, whatever the cell line that is used for production. The exact concentration of disaccharide (such as sucrose) or trisaccharide (such as raffinose) to be added in the cell culture medium, at a given osmolality will have to be determined case by case, depending on the performance of production and/or the glycosylation profile the skilled one wish to obtain molecule per molecule. This determination can be done without involving any inventive skill, based on the teaching of the present invention. The skilled person will also understand that he can use any disaccharide or trisaccharide, without being limited to raffinose or sucrose, in a culture medium having a constant osmolality, in any method for producing a protein such as an antibody, even if he does not aim to reach a particular glycosylation profile, but simply in order to improve the efficiency of at least one production run.

### REFERENCES

1) N. Yamane-Ohnuki et M. Satoh, 2009. Production of therapeutic antibodies with controlled fucosylation; mAbs, 1(3): 230-236
2) Yu et al., 2012. Characterization and pharmacokinetic properties of antibodies with N-linked Mannose-5 glycans”; mAbs, 4(4):475-487.
3) Cell Culture Technology for Pharmaceutical and Cell-Based Therapies, Sadettin Ozturk, Wei-Shou Hu, ed., CRC Press (2005)
4) Kim et al., 2004, Biotechnol. Prog., 20:1788-1796
5) Stettler et al., 2006. Biotechnol Bioeng. 95(6): 1228-1233
6) Ziv Roth et al., 2012. Identification and Quantification of Protein Glycosylation; International Journal of Carbohydrate Chemistry, Article ID 640923.
7) Ting Song et al., 2014. In-Depth Method for the Characterization of Glycosylation in Manufactured Recombinant Monoclonal Antibody Drugs; Anal. Chem., 86(12):5661-5666
8) Essentials of Glycobiology, Varki et al. eds., 1999, CSHL Press
9) Voisard et al., 2003, Biotechnol. Bioeng. 82:751-765
10) Ausubel et al., 1988 and updates, Current Protocols in Molecular Biology, eds. Wiley & Sons, New York.
11) Sambrook et al., 1989 and updates, Molecular Cloning: A Laboratory Manual, Cold Spring Laboratory Press.
12) Remington's Pharmaceutical Sciences, 1995, 18th ed., Mack Publishing Company, Easton, Pa.

1-16. (canceled)

17. A method of producing a recombinant protein in fed-batch or batch mode, said method comprising culturing a mammalian host cell expressing said recombinant protein in a cell culture medium comprising a dissacharide or a trisaccharide, or supplemented with a dissacharide or a trisaccharide, while maintaining the osmolality similar to the one of a standard medium which does not comprise said disaccharide or trisaccharide.

18. The method according to claim 17, wherein said method increases the efficiency of at least one production run.

19. The method according to claim 18, wherein the efficiency of a production run is measured by an increase of the viable cell density and/or a lower decrease in cell viability.

20. The method according to claim 17, wherein the disaccharide is sucrose and the trisaccharide is raffinose.

21. The method according to claim 17, wherein the host cell is Chinese Hamster Ovary (CHO) cells.

22. The method according to claim 17, wherein the recombinant protein is selected from the group consisting of a recombinant fusion protein, a growth factor, a hormone, a cytokine, an antibody or antigen binding fragment thereof, a human antibody or antigen-binding portion thereof, a humanized antibody or antigen-binding portion thereof, a chimeric antibody and antigen-binding portion thereof.

23. The method according to claim 17, wherein the concentration of disaccharide or trisaccharide in the cell culture medium is of about 0.1 mM to 100 mM.

24. A method of culturing in fed-batch or batch mode a mammalian host cell that expresses a recombinant protein, said method comprising culturing said host cell in a cell culture medium comprising a dissacharide or a trisaccharide, or supplemented with a dissacharide or a trisaccharide, while maintaining the osmolality similar to the one of a standard medium which does not comprise said disaccharide or trisaccharide.

25. A method of increasing production of a recombinant protein in fed-batch or batch mode, said method comprising culturing a mammalian host cell expressing said protein in a cell culture medium comprising a dissacharide or a trisaccharide, or supplemented with a dissacharide or a trisaccharide, while maintaining the osmolality similar to the one of a standard medium which does not comprise said disaccharide or trisaccharide.

26. A method of producing a recombinant protein with a modulated glycosylation profile, said method comprising culturing a host cell expressing said protein in cell culture medium comprising a disaccharide or a trisaccharide or supplemented with a disaccharide or a trisaccharide, while maintaining the osmolality of the culture medium similar to the one of a standard medium which does not comprise said disaccharide or trisaccharide.

27. The method according to claim 26, further comprising purifying said recombinant protein with a modulated glycosylation profile.

28. The method according to claim 26, wherein the modulated glycosylation profile of the protein comprises modulation of fucosylation level and/or mannosylation level in said protein.

29. The method according to claim 28, wherein the modulation of the fucosylation level is a decrease in the fucosylation level and wherein the modulation of the mannosylation level is an increase in the mannosylation level in said protein.

30. A method of producing a recombinant protein with a modulated glycosylation profile, said method comprising culturing a host cell expressing said protein in cell culture medium complemented with at least one feed comprising a disaccharide or a trisaccharide while maintaining the osmolality of the culture medium similar to the one of a standard medium which does not comprise said disaccharide or trisaccharide.
